(19) United States
(12) Patent Application Publication
(10) Pub. No.: US 2026/0191996 A1
(43) Pub. Date: Jul. 9, 2026

(54) METHODS FOR TREATING LIVER CANCER BY GENOMIC TARGETING

(71) Applicant: UNIVERSITY OF PITTSBURGH - OF THE COMMONWEALTH SYSTEM OF HIGHER EDUCATION, Pittsburgh, PA (US)

(72) Inventor: Jianhua LUO, Wexford, PA (US)

(73) Assignee: UNIVERSITY OF PITTSBURGH - OF THE COMMONWEALTH SYSTEM OF HIGHER EDUCATION, Pittsburgh, PA (US)

(21) Appl. No.: 19/578,344

(22) Filed: Mar. 25, 2026

Related U.S. Application Data
(63) Continuation of application No. PCT/US2024/047947, filed on Sep. 23, 2024.
(60) Provisional application No. 63/585,085, filed on Sep. 25, 2023.

(51) Int. Cl. A61K 48/00; A61K 31/522; A61P 35/00; C12N 9/12; C12N 9/22; C12N 15/11; C12N 15/90

(52) U.S. Cl. CPC A61K 48/005; A61K 31/522; A61P 35/00; C12N 9/1211; C12N 9/226; C12N 15/111; C12N 15/907; C12N 2310/20; C12Y 207/01021

(57) ABSTRACT

The present disclosure relates to methods of treating patients having liver cancer by performing a genome-targeting technique. It is based, at least in part, on the discovery that a genome editing technique that specifically targets genomic mutations can induce cell death in a cancer cell, e.g., a hepatocellular cancer cell, having the genomic gene. The present disclosure provides methods for treating cancer patients that include performing a genome editing technique targeting a genomic mutation present within one or more cells of a subject to produce an anti-cancer effect.

### CROSS-REFERENCES TO RELATED APPLICATIONS

This application is a continuation of International Patent Application No. PCT/US2024/047947, filed on Sep. 23, 2024, which claims the benefit of priority to U.S. Provisional Patent Application No. 63/585,085, filed on Sep. 25, 2023, the contents of each of which are incorporated herein by reference in their entireties, and to each of which priority is claimed.

### GRANT INFORMATION

This invention was made with government support under CA229262, CA251155, CA204586, and DK120531 awarded by the National Institutes of Health. The government has certain rights in the invention.

### SEQUENCE LISTING

This application contains a Sequence Listing which has been submitted electronically in XML format and is hereby incorporated by reference in its entirety. Said XML copy, created on Sep. 17, 2024, is named 072396.1032_ST26.xml, and is 38,753 bytes in size.

### 1. FIELD OF INVENTION

The present invention relates to methods of treating patients having liver cancer by performing a genome-targeting technique.

### 2. BACKGROUND

Liver cancer is one of the most lethal malignancies for humans. The treatment options for advanced-stage liver cancer remain limited. In 2022, 29,380 people in the United States died from liver cancer¹. Worldwide, mortality for liver cancer reached 830,180 in 2019². Hepatocellular carcinoma (HCC) has been the dominant liver cancer type, accounting for over 90% of all liver cancers. The most effective treatments for HCC are surgical resection and liver transplantation³. However, these options are limited to early-stage of liver cancer. For intermediate stages of HCC, transarterial radioembolization or transarterial chemoembolization has been employed with variable short-term successful rates. Long-term survival, however, remains elusive. For late-stage HCC, there is no effective treatment. The median survival time for late-stage HCC ranges from 6.1 months to 10.3 months⁴. Thus, effective treatment for unresectable HCC is urgently needed to reduce the mortality of the disease.

Numerous genetic alterations have been discovered in liver cancer cells, including single nucleotide mutations5, genome deletion/amplification^{6,7}, chromosome rearrangement, and gene fusion generation⁸⁻¹³. These genomic alterations underlie the development of HCC.

A previous study has shown that chromosome rearrangement in human cancers is targetable through Cas9 genomic editing¹⁴. By insertion of HSV1-tk gene into the breakpoint of fusion gene MAN2A1-FER or TMEM135-CCDC67 and the application of pro-drug ganciclovir, partial remission of xenografted cancers was achieved in mice.

The present disclosure provides methods for targeting single nucleotide mutations and gene fusions in the HCC genome to treat liver cancers. The present disclosure demonstrated that said treatment methods significantly reduced the tumor burden, decreased metastasis, and improved animal survival.

### 3. SUMMARY

The present disclosure provides a genome editing method comprising: (i) introducing a first polynucleotide encoding a suicide gene and a second polynucleotide encoding a nuclease into a cell, wherein the nuclease targets a genomic mutation of a gene selected from CTNNB1, SLTM, SLC45A2, AMACR, or a combination thereof, and (ii) contacting the cell with an agent capable of inducing killing of the cell.

In certain embodiments, the suicide gene is selected from herpes simplex virus thymidine kinase (HSV-tk), Exotoxin A from Pseudomonas aeruginosa, Diphtheria toxin from Corynebacterium diphtheri, Ricin or abrin from Ricinus communi (castor oil plant), Cytosine deaminase, Carboxyl esterase, Varicella Zoster virus (VZV) thymidine kinase, or a combination thereof. In certain embodiments, the suicide gene is herpes simplex virus thymidine kinase (HSV-tk). In certain embodiments, the suicide gene comprises an amino acid sequence at least 80% identical to the amino acid sequence set forth in SEQ ID NO: 25. In certain embodiments, the suicide gene comprises the amino acid sequence set forth in SEQ ID NO: 25.

In certain embodiments, the first polynucleotide comprises a first homology recombination arm and a second homology recombination arm. In certain embodiments, the first homology recombination arm and the second homology recombination arm flank the first polynucleotide.

In certain embodiments, the nuclease is selected from a zinc finger nuclease, a meganuclease, a TALE nuclease, or a CRISPR system nuclease. In certain embodiments, the nuclease is a CRISPR system nuclease. In certain embodiments, the nuclease is selected from Cas1, Cas1B, Cas2, Cas3, Cas4, Cas5, Cas6, Cas7, Cas8, Cas9 (also known as Csn1 or Csx12), Cas9^{D10A}, Cas1O, Csy1, Csy2, Csy3, Cse 1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, Cas9HiFi, or a combination thereof. In certain embodiments, the nuclease is Cas9 or Cas9^{D10A}. In certain embodiments, the method further comprises one or more guide RNAs (gRNAs). In certain embodiments, the one or more gRNAs targets the genomic mutation.

In certain embodiments, the genomic mutation is a single nucleotide mutation or a single nucleotide polymorphism (SNP). In certain embodiments, the genomic mutation is a mutation of an oncogene, a tumor suppressor gene, or an oncogenic fusion gene. In certain embodiments, the genomic mutation is a mutation of the CTNNB1 gene. In certain embodiments, the mutation of the CTNNB1 gene is a S45P mutation of CTNNB1. In certain embodiments, the genomic mutation is a mutation of the SLTM gene. In certain embodiments, the mutation of the SLTM gene is a V235G mutation of SLTM. In certain embodiments, the genomic mutation is a breakpoint of a SLC45A2-AMACR fusion gene. In certain embodiments, the genomic mutation is a mutation of the SLC45A2-AMACR fusion gene.

In certain embodiments, the first polynucleotide and the second polynucleotide are included in a vector. In certain embodiments, the first polynucleotide is included in a vector and the second polynucleotide is included in a second vector.

In certain embodiments, the agent is selected from ganciclovir, valganciclovir, or a combination thereof. In certain embodiments, the agent is ganciclovir.

The present disclosure also provides a genome editing system comprising: (i) a first polynucleotide encoding a suicide gene; (ii) a second polynucleotide encoding a nuclease targeting a genomic mutation of a gene selected from CTNNB1, SLTM, SLC45A2, AMACR, or a combination thereof, and (iii) an agent capable of inducing killing of a cell expressing the suicide gene.

In certain embodiments, the suicide gene is selected from herpes simplex virus thymidine kinase (HSV-tk), inducible Caspase 9 suicide gene (iCasp-9), truncated human epidermal growth factor receptor (EGFRt), or a combination thereof. In certain embodiments, the suicide gene is herpes simplex virus thymidine kinase (HSV-tk). In certain embodiments, the suicide gene comprises an amino acid sequence at least 80% identical to the amino acid sequence set forth in SEQ ID NO: 25. In certain embodiments, the suicide gene comprises the amino acid sequence set forth in SEQ ID NO: 25.

In certain embodiments, the first polynucleotide comprises a first homology recombination arm and a second homology recombination arm. In certain embodiments, the first homology recombination arm and the second homology recombination arm flank the first polynucleotide.

In certain embodiments, the nuclease is selected from a zinc finger nuclease, a meganuclease, a TALE nuclease, or a CRISPR system nuclease. In certain embodiments, the nuclease is a CRISPR system nuclease. In certain embodiments, the nuclease is selected from Cas1, Cas1B, Cas2, Cas3, Cas4, Cas5, Cas6, Cas7, Cas8, Cas9 (also known as Csn1 or Csx12), Cas9^{D10A}, Cas1O, Csy1, Csy2, Csy3, Cse 1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, Cas9HiFi, or a combination thereof. In certain embodiments, the nuclease is Cas9 or Cas9^{D10A}. In certain embodiments, the system further comprises one or more guide RNAs (gRNAs). In certain embodiments, the one or more gRNAs targets the genomic mutation. In certain embodiments, the genomic mutation is a single nucleotide mutation or a single nucleotide polymorphism (SNP). In certain embodiments, the genomic mutation is a mutation of an oncogene, a tumor suppressor gene, or an oncogenic fusion gene.

In certain embodiments, the genomic mutation is a mutation of the CTNNB1 gene. In certain embodiments, the mutation of the CTNNB1 gene is a S45P mutation of CTNNB1. In certain embodiments, the genomic mutation is a mutation of the SLTM gene. In certain embodiments, the mutation of the SLTM gene is a V235G mutation of SLTM. In certain embodiments, the genomic mutation is a breakpoint of a SLC45A2-AMACR fusion gene. In certain embodiments, the genomic mutation is a mutation of the SLC45A2-AMACR fusion gene.

In certain embodiments, the first polynucleotide and the second polynucleotide are included in a vector. In certain embodiments, the first polynucleotide is included in a vector and the second polynucleotide is included in a second vector. In certain embodiments, the agent is selected from ganciclovir, valganciclovir, or a combination thereof. In certain embodiments, the agent is ganciclovir.

Additionally, the present disclosure provides a composition comprising the genome editing system disclosed herein. In certain embodiments, the composition is a pharmaceutical composition. Moreover, the present disclosure provides a kit comprising the genome editing system or the composition disclosed herein.

Further, the present disclosure provides methods of treating a subject having a pre-malignant or neoplastic condition, treating a subject having a cancer, preventing, minimizing, and/or reducing the growth of a cancer in a subject, lengthening the period of survival of a subject having cancer, or reducing the risk of, inhibiting, or preventing metastatic spread of a cancer in a subject. In certain embodiments, the methods comprise detecting, in a sample obtained from the subject, a genomic mutation of a gene selected from CTNNB1, SLTM, SLC45A2, AMACR, or a combination thereof. In certain embodiments, the methods comprise administering an effective amount of a first polynucleotide encoding a suicide gene and a second polynucleotide encoding a nuclease targeting the genomic mutation. In certain embodiments, the methods further comprise administering an effective amount of an agent capable of inducing killing of a cell expressing the suicide gene.

In certain embodiments, the pre-malignant or neoplastic condition is a condition of the liver. In certain embodiments, the condition of the liver is hepatocellular carcinoma (HCC). In certain embodiments, the cancer is a liver cancer. In certain embodiments, the liver cancer is hepatocellular carcinoma (HCC).

In certain embodiments, the genomic mutation is a single nucleotide mutation or a single nucleotide polymorphism (SNP). In certain embodiments, the genomic mutation is a mutation of an oncogene, a tumor suppressor gene, or an oncogenic fusion gene. In certain embodiments, the genomic mutation is a mutation of the CTNNB1 gene. In certain embodiments, the mutation of the CTNNB1 gene is a S45P mutation of CTNNB1. In certain embodiments, the genomic mutation is a mutation of the SLTM gene. In certain embodiments, the mutation of the SLTM gene is a V235G mutation of SLTM. In certain embodiments, the genomic mutation is a breakpoint of a SLC45A2-AMACR fusion gene. In certain embodiments, the genomic mutation is a mutation of the SLC45A2-AMACR fusion gene. In certain embodiments, the agent is selected from ganciclovir, valganciclovir, or a combination thereof.

Finally, the present disclosure provides compositions or kits disclosed herein for use in treating a subject having a pre-malignant or neoplastic condition, treating a subject having a cancer, preventing, minimizing, and/or reducing the growth of a cancer in a subject, lengthening the period of survival of a subject having cancer, or reducing the risk of, inhibiting, or preventing metastatic spread of a cancer in a subject; and/or use of compositions or kits disclosed herein for the manufacturing of a medicament for treating a subject having a pre-malignant or neoplastic condition, treating a subject having a cancer, preventing, minimizing, and/or reducing the growth of a cancer in a subject, lengthening the period of survival of a subject having cancer, or reducing the risk of, inhibiting, or preventing metastatic spread of a cancer in a subject.

### 4. BRIEF DESCRIPTION OF THE DRAWINGS

For a more complete understanding of the present disclosure and its features and advantages, reference is now made to the following description, taken in conjunction with the accompanying drawings.

FIGS. 1A-1C show the targeting of the S45P mutation of CTNNB1. FIG. 1A shows the gRNA design and in vitro cleavage of mutant DNA by SpCas9. The top panel of FIG. 1A shows a sequence with C. 133T>C mutation from exon 2 of CTNNB1. gRNA design is indicated by a rectangle bracket (SEQ ID NOs: 26 and 27). The mutation position is indicated in red. The corresponding wild-type nucleotide is indicated. The bottom panel of FIG. 1A shows the cleavage of mutant DNA by gRNA-, while the cleavage of WT DNA by gRNA-is negligible. FIG. 1B shows HUH7 cells transformed with CTNNB1^{S45P} showed insertion/expression of mCherry-HSV1-tk, while HUH7 cells transformed with CTNNB1 showed no insertion/expression when treated with ad-Cas9^{D10A}-EGFP/ad-CTNNB1-mCherry-tk-gRNA. The top panels of FIG. 1B show diagrams of targeting constructs. The bottom panel of FIG. 1B shows representative images of EGFP and mCherry fluorescence. FIG. 1C shows the induction of expression of mCherry-HSV1-tk in HUH7 cells transformed with CTNNB1S45P but not with CTNNB1, as assessed by flow cytometry analysis.

FIGS. 2A-2F show the therapeutic effect of targeting S45P of CTNNB1. FIG. 2A shows representative magnetic resonance images of CTNNB1^{S45P}/HMET-induced liver cancer.

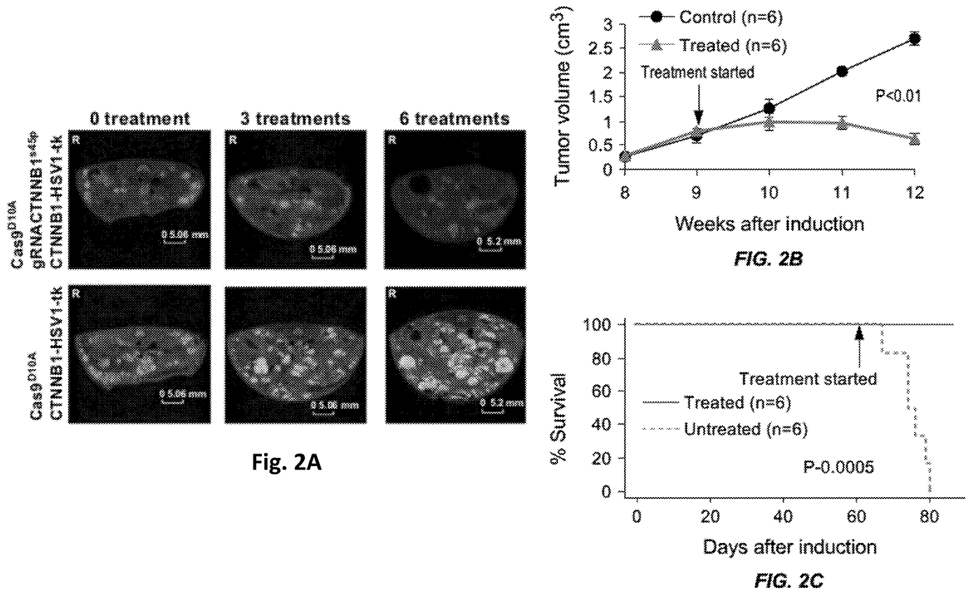

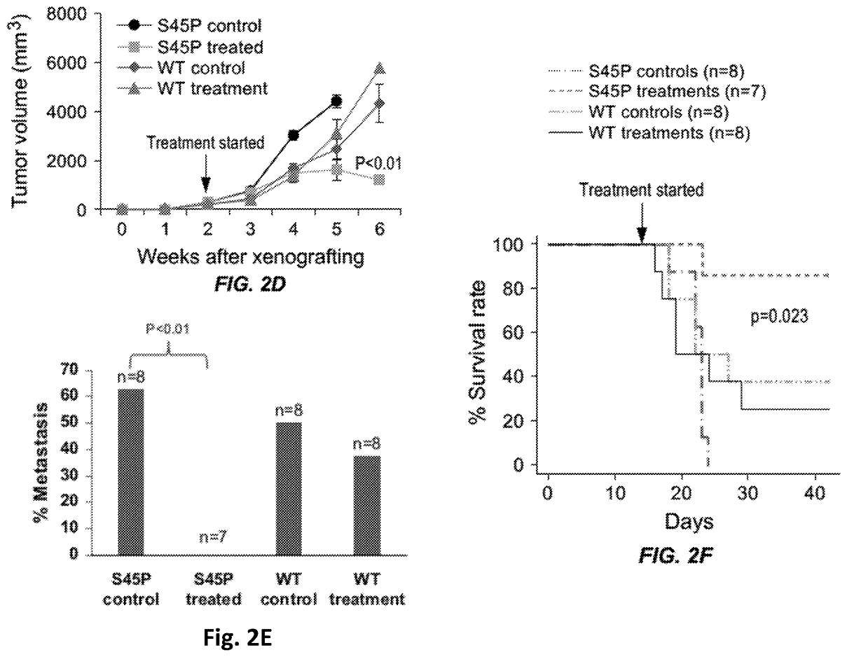

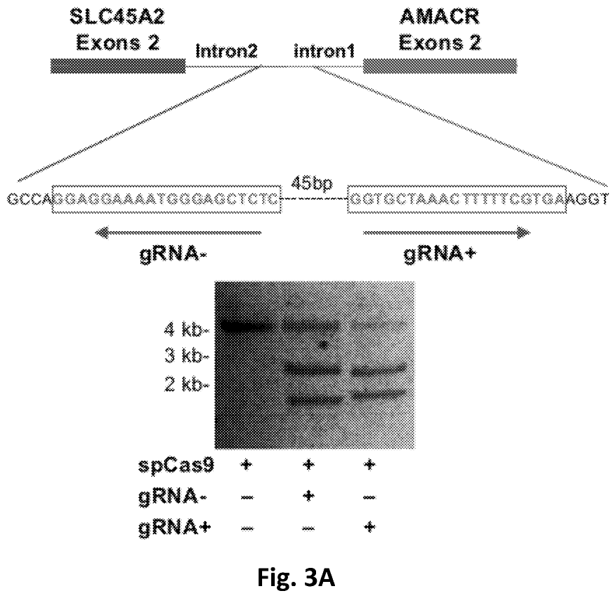

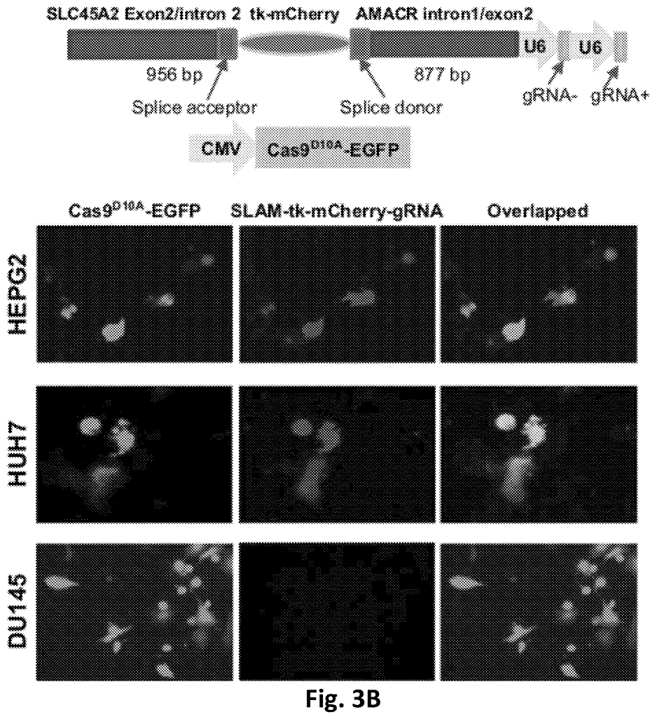

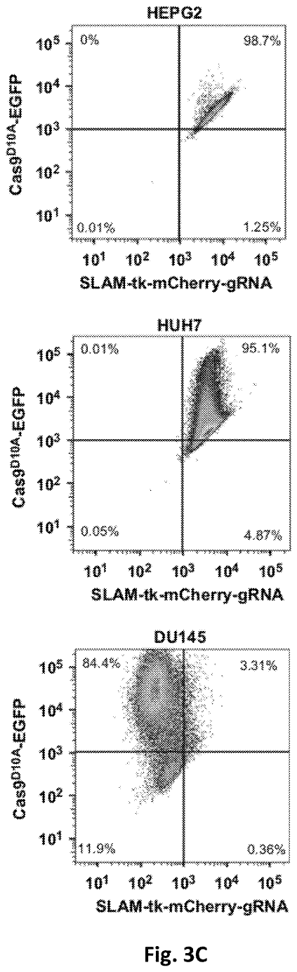

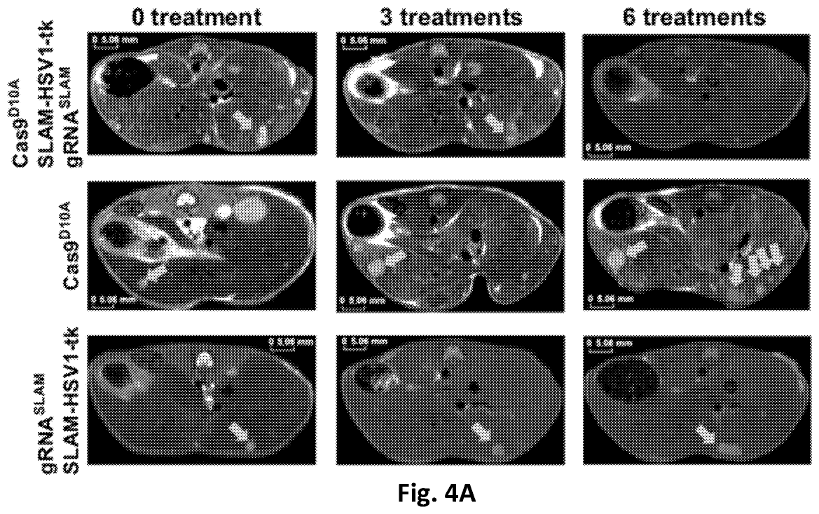

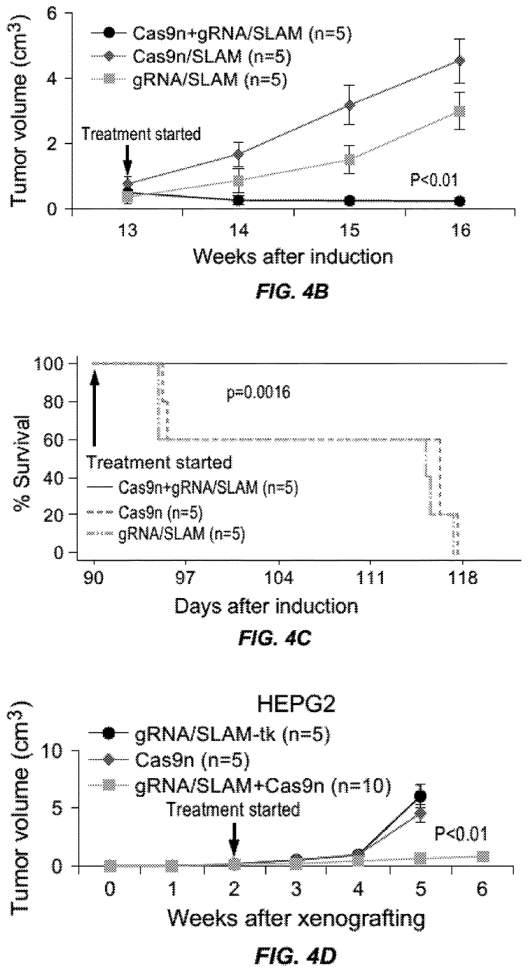

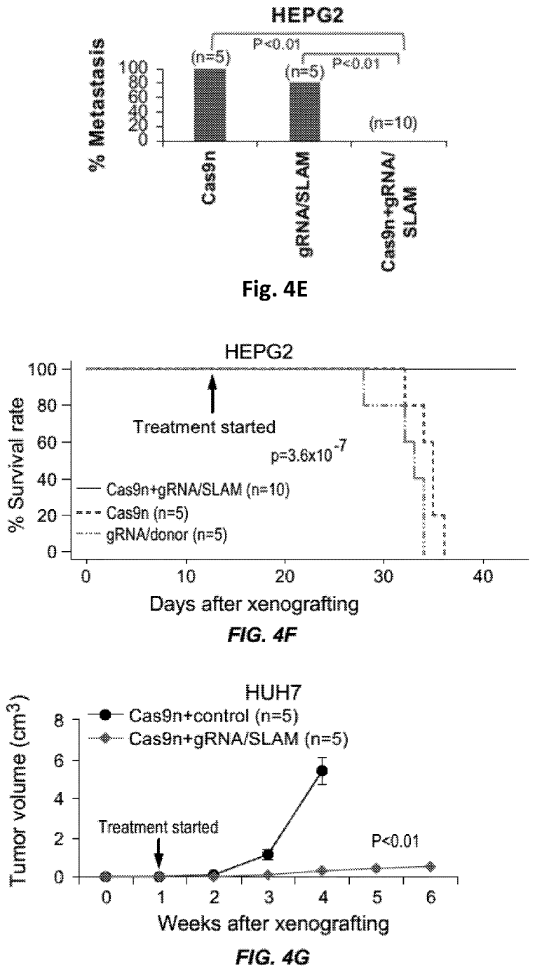

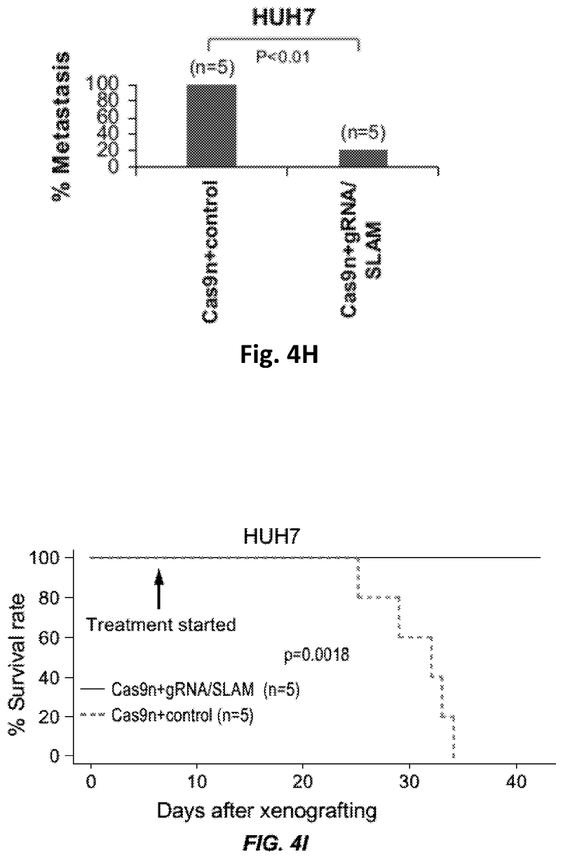

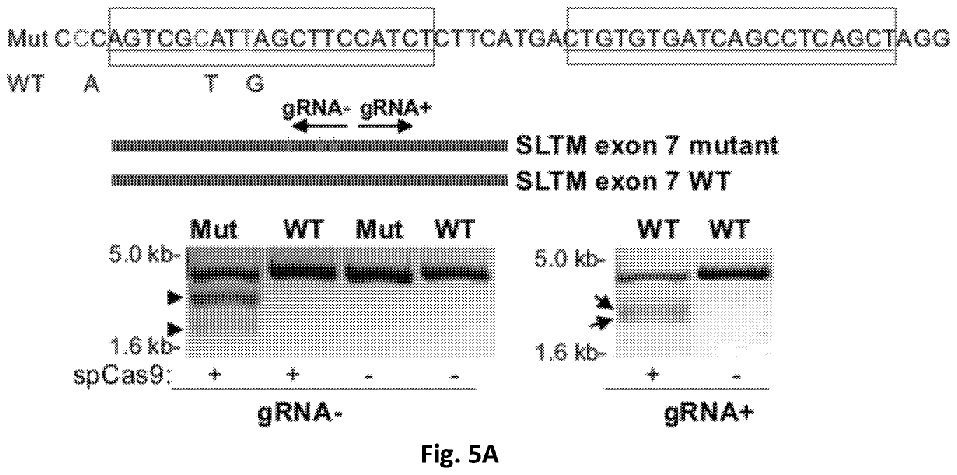

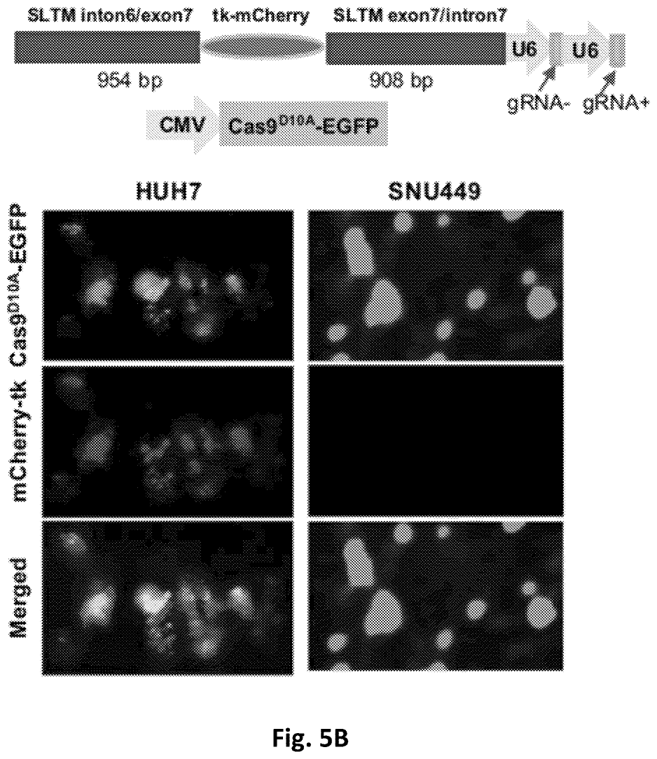

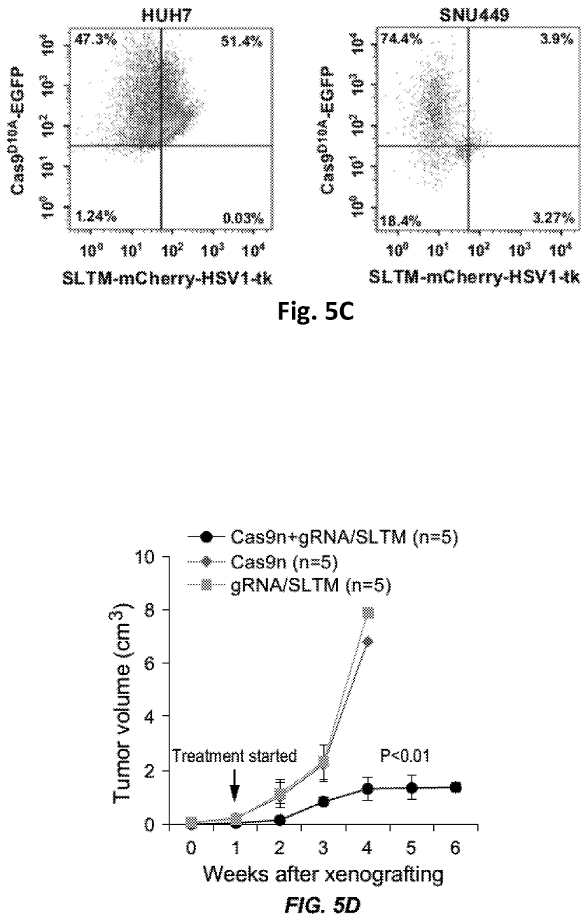

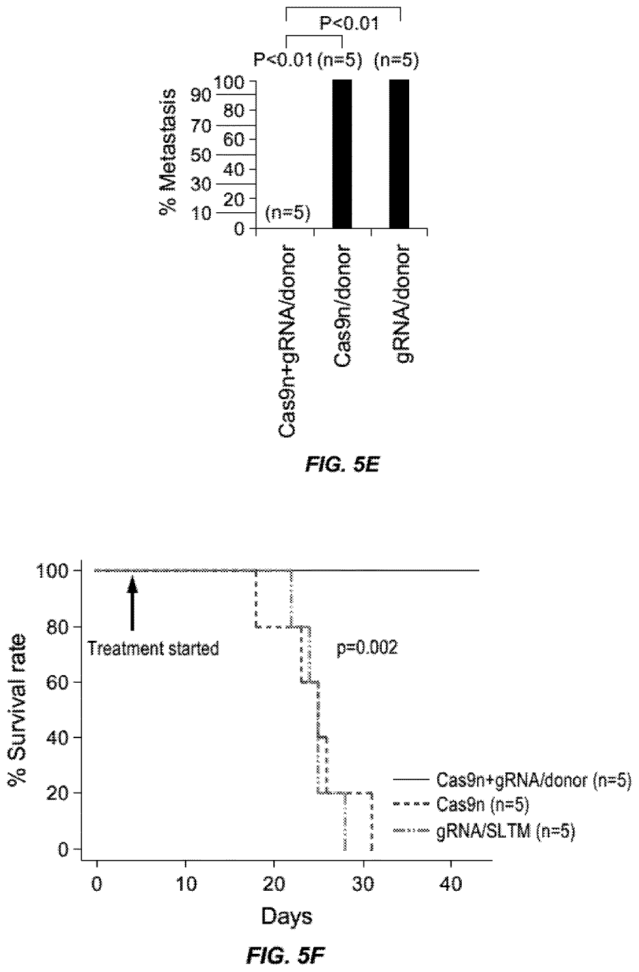

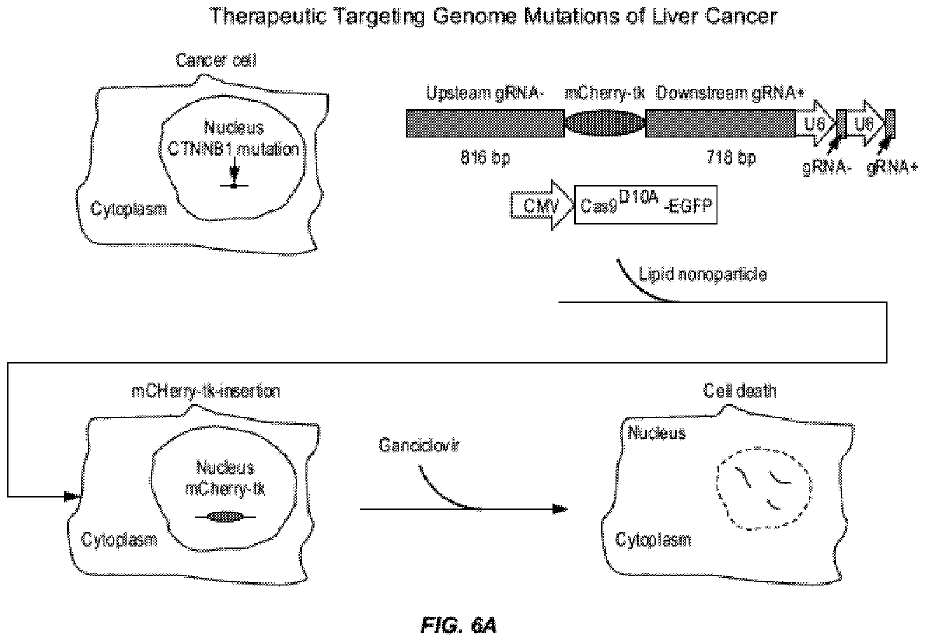

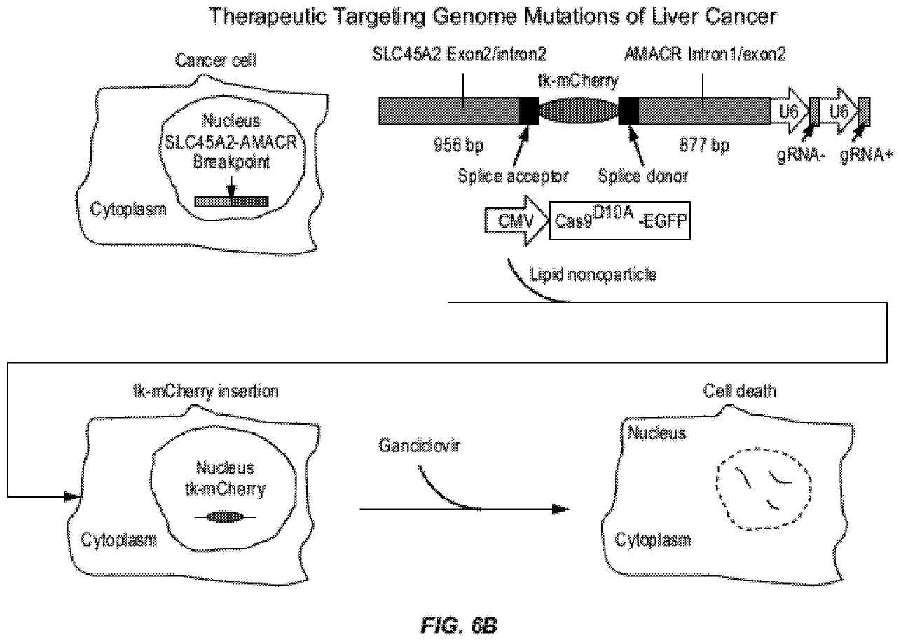

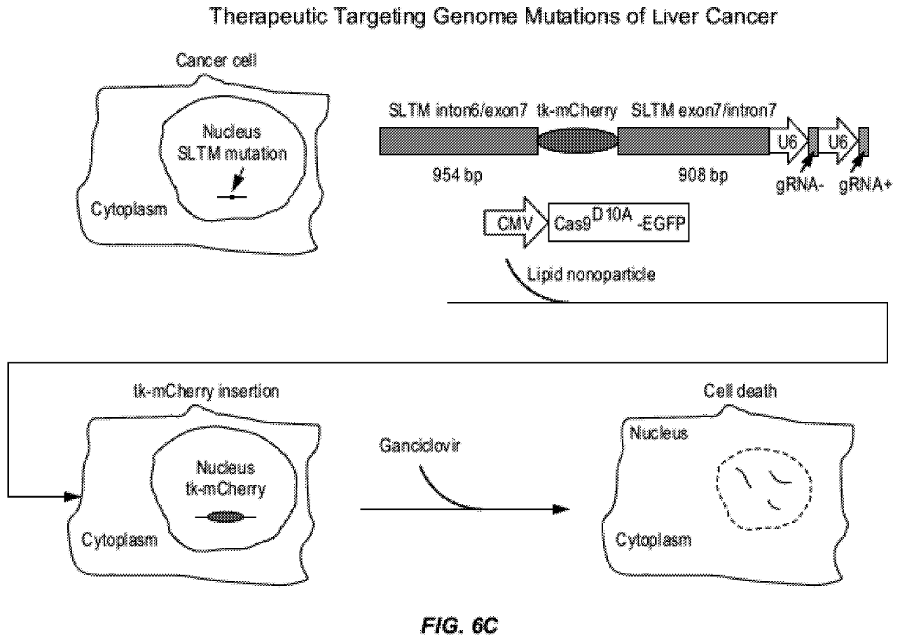

The top panel of FIG. 2A shows representative MRI images of a mouse treated with pCas9^{D10A}-EGFP/pCTNNB1-mCherry-tk-gRNA (treated). The bottom panel of FIG. 2A shows representative MRI images of a mouse treated with pCas9^{D10A}-EGFP/pCTNNB1-mCherry-tk (control). FIG. 2B shows accumulated liver cancer growth by image analysis of treated and control groups. FIG. 2C shows a Kaplan-Mier analysis of mice survival from the treatment and control groups. FIG. 2D shows treatment targeting S45P of CTNNB1 reduced the tumor burden of xenografted HUH7-CTNNB1^{S45P} but not HUH7-CTNNB1 in SCID mice. FIG. 2E shows treatment targeting S45P of CTNNB1 reduced metastasis of xenografted HUH7-CTNNB1^{S45P} in SCID mice. FIG. 2F shows treatment targeting S45P of CTNNB1 reduced the mortality of SCID mice xenografted with HUH7-CTNNB1^{S45P} cells.

FIGS. 3A-3C show the targeting of the chromosome breakpoint of SLC45A2-AMACR. FIG. 3A shows the gRNA design and in vitro cleavage of mutant DNA by SpCas9. The top panel of FIG. 3A shows the diagram of the breakpoint region between SLC45A2 intron 2 and AMACR intron 1. gRNA design is indicated in red enclosed by a rectangle. The bottom panel of FIG. 3A shows the cleavage of the sequence containing the breakpoint by spCas9 with gRNA+ or gRNA−. FIG. 3B shows SLC45A2-AMACR positive HUH7 and HEPG2 cells with insertion/expression of HSV1-tk-mCherry, while SLC45A2-AMACR negative DU145 cells showed no expression of HSV1-tk-mCherry when treated with ad-Cas9^{D10A}-EGFP/ad-SLAM-mCherry-tk-gRNA. The top panel of FIG. 3B shows diagrams of targeting constructs. The bottom panel of FIG. 3B shows representative images of EGFP and mCherry fluorescence. FIG. 3C shows flow cytometry analysis of HEPG2, HUH7, and DU145 cells when treated with ad-Cas9^{D10A}-EGFP/ad-SLAM-mCherry-tk-gRNA.

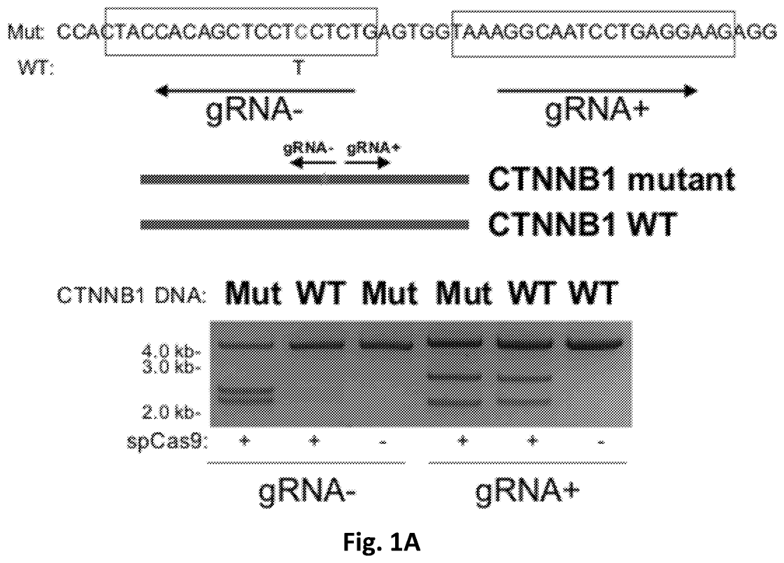

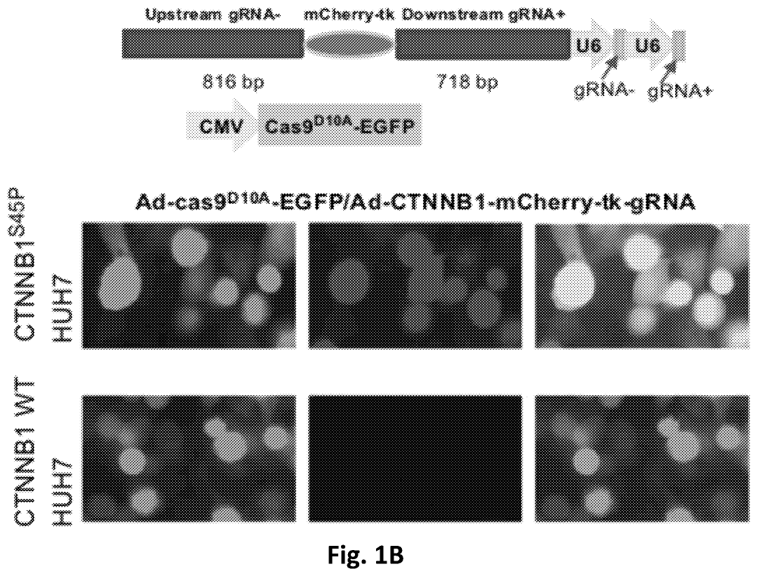

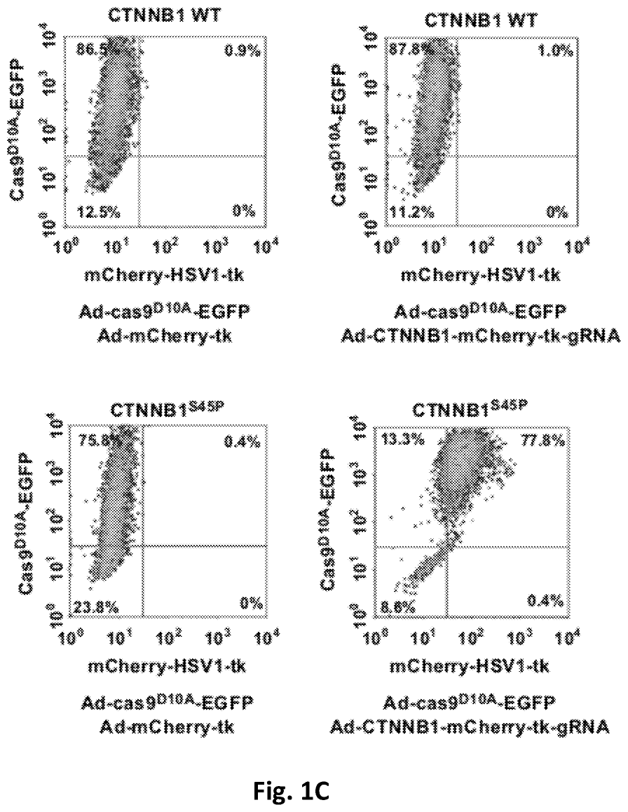

FIGS. 4A-4I shows the therapeutic effect of targeting the chromosome breakpoint of SLC45A2-AMACR gene fusion. FIG. 4A shows magnetic resonance images of SLC45A2-AMACR/Pten knockout-induced liver cancer. The top panel of FIG. 1A shows representative MR images of a mouse treated with pCas9^{D10A}-EGFP/pSLAM-tk-mCherry-gRNA (treated). The middle panel of FIG. 4A shows representative MR images of a mouse treated with pCas9^{D10A}-EGFP (control). The bottom panel of FIG. 4A shows representative MR images of a mouse treated with pSLAM-tk-mCherry-gRNA (control). Green arrows indicate the position of liver cancer in the image. FIG. 4B shows the accumulated liver cancer growth by image analysis of treated (Cas9n+gRNA/SLAM) and control groups (Cas9n or gRNA/SLAM). FIG. 4C shows a Kaplan-Mier analysis of mice survival after treatment (Cas9n+gRNA/SLAM) or control treatment (Cas9n or gRNA/SLAM). FIG. 4D shows the treatment targeting the chromosome breakpoint of SLC45A2-AMACR reduced the tumor burden of HEPG2 xenografted tumor in SCID mice. FIG. 4E shows treatment targeting the chromosome breakpoint of SLC45A2-AMACR reduced the rate of metastasis of HEPG2 xenografted tumors in SCID mice. FIG. 4F shows treatment targeting the chromosome breakpoint of SLC45A2-AMACR reduced mortality of mice xenografted with HEPG2 tumor. FIG. 4G shows treatment targeting the chromosome breakpoint of SLC45A2-AMACR reduced the tumor burden of HUH7 xenografted tumor in SCID mice. FIG. 4H shows treatment targeting the chromosome breakpoint of SLC45A2-AMACR reduced the rate of metastasis of HUH7 xenografted tumors in SCID mice. FIG. 4I shows treatment targeting the chromosome breakpoint of SLC45A2-AMACR reduced mortality of mice xenografted with HUH7 tumor.

FIGS. 5A-5F show the targeting of the V235G mutation of SLTM in vitro and in vivo. FIG. 5A shows the gRNA design and in vitro cleavage of mutant DNA by SpCas9. The top panel of FIG. 5A shows the sequence with mutations (Hg19-Chr15: 58899823, Hg19-Chr15: 58899830, and Hg19-Chr15: 58899833) in exon 7 of SLTM. gRNA design is indicated by a rectangle bracket. The mutation position is indicated in red. The corresponding wild-type nucleotide is indicated. The bottom panel of FIG. 5A shows the cleavage of mutant DNA by gRNA−, while cleavage of WT DNA by gRNA-is negligible. Arrows indicate cleaved DNA fragments of the correct sizes. FIG. 5B shows HUH7 cells having insertion/expression of mCherry-HSV1-tk, while SNU449, which was negative for the mutation, showed no insertion/expression when treated with ad-Cas9^{D10A}-EGFP/pSLTM-mCherry-tk-gRNA. The top panel of FIG. 5B shows diagrams of targeting constructs. The bottom panel of FIG. 5B shows representative images of EGFP and mCherry fluorescence. FIG. 5C shows the induction of expression of mCherry-HSV1-tk in HUH7 cells but not SNU449 cells. FIG. 5D shows treatment targeting V235G of SLTM reduced the tumor burden of xenografted HUH7. FIG. 5E shows treatment targeting V235G of SLTM reduced metastasis of xenografted HUH7 in SCID mice. FIG. 5F shows treatment targeting V235G of SLTM reduced mortality of SCID mice xenografted with HUH7 cells.

FIGS. 6A-6C show a schematic illustrating the mechanism-of-action of the presently disclosed genome editing systems and genome editing methods. Therapeutic targeting of genome mutation of liver cancer can be directed to cancer cells (e.g., liver cells) expressing one of the presently disclosed mutations of CTNNB1, SLC45A2-AMACR, or SLTM (FIG. 6A, FIG. 6B, and FIG. 6C, respectively). Use of vectors (e.g., lipid nanoparticles) and ganciclovir allows the delivery of the suicide gene HSV-tk and its apoptosis-inducing activity, respectively.

### 5. DETAILED DESCRIPTION

The present disclosure relates to methods of treating patients having liver cancer by performing a genome-targeting technique. For clarity, and not by way of limitation, the detailed description of the invention is divided into the following subsections:
5.1 Definitions;
5.2 Genome Mutations;
5.3 Detecting Genome Mutations;
5.4 Cancer Targets;
5.5 Methods of Treatment;
5.6 Genome Targeting/Editing Techniques;
5.7 Kits; and
5.8 Exemplary Embodiments.

### 5.1 Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the disclosure and how to make and use them.

As used herein, the use of the word “a” or “an” when used in conjunction with the term “comprising” in the claims and/or the specification can mean “one,” but it is also consistent with the meaning of “one or more,” “at least one,” and “one or more than one.”

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other embodiments “comprising,” “consisting of”, and “consisting essentially of,” the embodiments or elements presented herein, whether explicitly set forth or not.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

An “individual” or “subject” herein is a vertebrate, such as a human or non-human animal, for example, a mammal. Mammals include, but are not limited to, humans, non-human primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” refers to any condition or disorder that damages or interferes with the normal function of a cell, tissue, or organ.

As used herein, the term “tumor,” refers to all neoplastic cell growth and proliferation, whether malignant or benign, and all pre-cancerous and cancerous cells and tissues.

The term “nucleic acid molecule” and “nucleotide sequence,” as used herein, refers to a single or double-stranded covalently-linked sequence of nucleotides in which the 3′ and 5′ ends on each nucleotide are joined by phosphodiester bonds. The nucleic acid molecule can include deoxyribonucleotide bases or ribonucleotide bases, and can be manufactured synthetically in vitro or isolated from natural sources.

As used herein, the term “agent” means a substance that produces or is capable of producing an effect and would include, but is not limited to, chemicals, pharmaceuticals, biologics, small organic molecules, antibodies, nucleic acids, peptides and proteins.

As used herein, the terms “inhibiting,” “eliminating,” “decreasing,” “reducing” or “preventing,” or any variation of these terms includes any measurable decrease or complete inhibition to achieve a desired result.

As used herein, the term “in need thereof” would be a subject known or suspected of having or being at risk of developing a disease, e.g., cancer.

As used herein, the term “disease” refers to any condition or disorder that damages or interferes with the normal function of a cell, tissue, or organ.

As used herein, the terms “detection” or “detecting” include any means of detecting, including direct and indirect detection.

### 5.2 Genome Mutations

The terms “genomic alteration,” “genomic mutation,” or “gene mutation” encompass a wide array of modifications in the genetic material. In this context, the term “mutation” as used herein, refers to any and all types of functional and/or non-functional nucleic acid changes, including mutations and polymorphisms in the target nucleic acid molecule when compared to a wildtype variant of the same nucleic acid region or allele or the more common nucleic acid molecule present on the sample. Such changes include but are not limited to, deletions, amplifications, insertions, translocations, inversions, and base substitutions of one or more nucleotides.

The term “single nucleotide mutation”, as used herein, refers to a DNA base within an established nucleotide sequence that differs from the known reference sequences. Single nucleotide mutation may be found within a patient sample (e.g., a tumor), they may or may not be present in unperturbed populations, and they include naturally occurring single nucleotide polymorphisms, also referred to as “SNPs”.

The term “single nucleotide polymorphism” or “SNP”, as used herein, refers to a DNA sequence variation occurring when a single nucleotide—A, T, C, or G—in the genome (or other shared sequence) differs between members of a species (or between paired chromosomes in an individual). Single nucleotide polymorphism refers to a single nucleotide position in a genomic sequence for which two or more alternative alleles are present at an appreciable frequency (e.g., at least 1%) in a population of organisms.

The term “fusion gene,” as used herein, refers to a nucleic acid or protein sequence which combines elements of the recited genes or their RNA transcripts in a manner not found in the wild-type/normal nucleic acid or protein sequences. For example, but not by way of limitation, in a fusion gene in the form of genomic DNA, the relative positions of portions of the genomic sequences of the recited genes are altered relative to the wild type/normal sequence (for example, as reflected in the NCBI chromosomal positions or sequences set forth herein). In a fusion gene in the form of mRNA, portions of RNA transcripts arising from both component genes are present (not necessarily in the same register as the wild-type transcript and possibly including portions normally not present in the normal mature transcript). In non-limiting embodiments, such a portion of genomic DNA or mRNA may comprise at least about 10 consecutive nucleotides, or at least about 20 consecutive nucleotides, or at least about 30 consecutive nucleotides, or at least 40 consecutive nucleotides. In certain embodiments, such a portion of genomic DNA or mRNA may comprise up to about 10 consecutive nucleotides, up to about 50 consecutive nucleotides, up to about 100 consecutive nucleotides, up to about 200 consecutive nucleotides, up to about 300 consecutive nucleotides, up to about 400 consecutive nucleotides, up to about 500 consecutive nucleotides, up to about 600 consecutive nucleotides, up to about 700 consecutive nucleotides, up to about 800 consecutive nucleotides, up to about 900 consecutive nucleotides, up to about 1,000 consecutive nucleotides, up to about 1,500 consecutive nucleotides or up to about 2,000 consecutive nucleotides of the nucleotide sequence of a gene present in the fusion gene. In certain embodiments, such a portion of genomic DNA or mRNA may comprise no more than about 10 consecutive nucleotides, about 50 consecutive nucleotides, about 100 consecutive nucleotides, about 200 consecutive nucleotides, about 300 consecutive nucleotides, about 400 consecutive nucleotides, about 500 consecutive nucleotides, about 600 consecutive nucleotides, about 700 consecutive nucleotides, about 800 consecutive nucleotides, about 900 consecutive nucleotides, about 1,000 consecutive nucleotides, about 1,500 consecutive nucleotides or about 2,000 consecutive nucleotides of the nucleotide sequence of a gene present in the fusion gene. In certain embodiments, such a portion of genomic DNA or mRNA does not comprise the full wildtype/normal nucleotide sequence of a gene present in the fusion gene. In a fusion gene in the form of a protein, portions of amino acid sequences arising from both component genes are present (not by way of limitation, at least about 5 consecutive amino acids or at least about 10 amino acids or at least about 20 amino acids or at least about 30 amino acids). In certain embodiments, such a portion of a fusion gene protein may comprise up to about 10 consecutive amino acids, up to about 20 consecutive amino acids, up to about 30 consecutive amino acids, up to about 40 consecutive amino acids, up to about 50 consecutive amino acids, up to about 60 consecutive amino acids, up to about 70 consecutive amino acids, up to about 80 consecutive amino acids, up to about 90 consecutive amino acids, up to about 100 consecutive amino acids, up to about 120 consecutive amino acids, up to about 140 consecutive amino acids, up to about 160 consecutive amino acids, up to about 180 consecutive amino acids, up to about 200 consecutive amino acids, up to about 220 consecutive amino acids, up to about 240 consecutive amino acids, up to about 260 consecutive amino acids, up to about 280 consecutive amino acids or up to about 300 consecutive amino acids of the amino acid sequence encoded by a gene present in the fusion gene. In certain embodiments, such a portion of a fusion gene protein may comprise no more than about 10 consecutive amino acids, about 20 consecutive amino acids, about 30 consecutive amino acids, about 40 consecutive amino acids, about 50 consecutive amino acids, about 60 consecutive amino acids, about 70 consecutive amino acids, about 80 consecutive amino acids, about 90 consecutive amino acids, about 100 consecutive amino acids, about 120 consecutive amino acids, about 140 consecutive amino acids, about 160 consecutive amino acids, about 180 consecutive amino acids, about 200 consecutive amino acids, about 220 consecutive amino acids, about 240 consecutive amino acids, about 260 consecutive amino acids, about 280 consecutive amino acids or about 300 consecutive amino acids of the amino acid sequence encoded by a gene present in the fusion gene. In certain embodiments, such a portion of a fusion gene protein does not comprise the full wildtype/normal amino acid sequence encoded by a gene present in the fusion gene. In this paragraph, portions arising from both genes, transcripts or proteins do not refer to sequences which may happen to be identical in the wild type forms of both genes (that is to say, the portions are “unshared”). As such, a fusion gene represents, generally speaking, the splicing together or fusion of genomic elements not normally joined together. See WO 2015/103057 and WO 2016/011428, the contents of which are hereby incorporated by reference, for additional information regarding the disclosed fusion genes.

In certain embodiments, the fusion gene is an oncogenic fusion gene. Oncogenic fusion genes are genes or DNA segments that translate into a fusion protein that alters the activity of other genes, leading to uncontrolled cell growth and tumor formation and are considered to drive events or changes leading to cancer or impairing the biological behavior (e.g., metastasizing) of a cancer cell.

In certain embodiments, a genomic mutation occurs in the CTNNB1 gene. The gene CTNNB1 encodes the protein beta-catenin (β-catenin), also known as catenin beta-1. The human CTNNB1 gene is typically located on human chromosome 3 (3p22.1). In certain embodiments, the CTNNB1 gene is the human gene having NCBI Gene ID No: 1499, sequence chromosome 3; NC_00003.12 (41199505.. 41240443). In certain embodiments, the mutation of the CTNNB1 gene encodes a S45P mutation of a CTNNB1 polypeptide.

In certain embodiments, a genomic mutation occurs in the SLTMgene. The gene SLTM encodes the protein SAFB-like transcription modulator. The human SLTM gene is typically located on human chromosome 15 (15q22.1). In certain embodiments, the SLTM gene is the human gene having NCBI Gene ID No: 79811, sequence chromosome 15; NC_000015.10 (58879050..58933679, complement). In certain embodiments, the mutation of the SLTMgene encodes a V235G mutation of a SLTM polypeptide.

In certain embodiment, a genomic mutation results in a SLC45A2-AMACR fusion gene. The fusion gene SLC45A2-AMACR is a fusion between the solute carrier family 45, member 2 (“SLC45A2”) and alpha-methylacyl-CoA racemase (“AMACR”) genes. The human SLC45A2 gene is typically located on human chromosome 5p13.2 and the human AMACR gene is typically located on chromosome 5p13. In certain embodiments the SLC45A2 gene is the human gene having NCBI Gene ID No: 51151, sequence chromosome 5; NC_000005.9 (33944721..33984780, complement) and/or the AMACR gene is the human gene having NCBI Gene ID No: 23600, sequence chromosome 5; NC_000005.9 (33987091..34008220, complement). In certain embodiments, the genomic mutation is a breakpoint of a SLC45A2-AMACR fusion gene.

### 5.3 Detecting Genome Mutations

Any of the described genomic mutations above in section 5.2 may be identified and/or detected by methods known in the art. Genomic mutations may be detected by detecting a mutation manifested in a DNA molecule, an RNA molecule or a protein. In certain embodiments, a genomic mutation can be detected by determining the presence of a DNA molecule, an RNA molecule or protein that is encoded by gene harboring a mutation. For example, and not by way of limitation, the presence of a genomic mutation may be detected by determining the presence of the protein encoded by a gene harboring a mutation.

The mutated gene may be detected in a sample of a subject. A “patient” or “subject,” as used interchangeably herein, refers to a human or a non-human subject. Non-limiting examples of non-human subjects include non-human primates, dogs, cats, mice, etc. The subject may or may not be previously diagnosed as having cancer.

In certain non-limiting embodiments, a sample includes, but is not limited to, cells in culture, cell supernatants, cell lysates, serum, blood plasma, biological fluid (e.g., blood, plasma, serum, stool, urine, lymphatic fluid, ascites, ductal lavage, saliva and cerebrospinal fluid) and tissue samples. The source of the sample may be solid tissue (e.g., from a fresh, frozen, and/or preserved organ, tissue sample, biopsy, or aspirate), blood or any blood constituents, bodily fluids (such as, e.g., urine, lymph, cerebral spinal fluid, amniotic fluid, peritoneal fluid or interstitial fluid), or cells from the individual, including circulating cancer cells. In certain non-limiting embodiments, the sample is obtained from a cancer. In certain embodiments, the sample may be a “biopsy sample” or “clinical sample,” which are samples derived from a subject. In certain embodiments, the sample includes one or more cancer cells from a subject. In certain embodiments, the mutated gene(s) can be detected in one or more samples obtained from a subject, e.g., in one or more cancer cell samples. In certain embodiments, the sample is a blood sample, e.g., buffy coat sample, from a subject.

In certain non-limiting embodiments, the genomic mutation is detected by nucleic acid hybridization analysis.

In certain non-limiting embodiments, the genomic mutation is detected by fluorescent in situ hybridization (FISH) analysis. FISH is a technique that can directly identify a specific sequence of DNA or RNA in a cell or biological sample and enables visual determination of the presence and/or expression of a mutated gene in a tissue sample. In certain non-limiting embodiments, where a genomic mutation combines genes not typically present on the same chromosome, FISH analysis may demonstrate probes binding to the same chromosome. For example, and not by way of limitation, analysis may focus on the chromosome where one gene normally resides and then hybridization analysis may be performed to determine whether the other gene is present on that chromosome as well.

In certain non-limiting embodiments, the genomic mutation is detected by DNA hybridization, such as, but not limited to, Southern blot analysis.

In certain non-limiting embodiments, the genomic mutation is detected by RNA hybridization, such as, but not limited to, Northern blot analysis. In certain embodiments, Northern blot analysis can be used for the detection of a mutated gene, where an isolated RNA sample is run on a denaturing agarose gel, and transferred to a suitable support, such as activated cellulose, nitrocellulose or glass or nylon membranes. Radiolabeled cDNA or RNA is then hybridized to the preparation, washed and analyzed by autoradiography to detect the presence of a mutated gene in the RNA sample.

In certain non-limiting embodiments, the genomic mutation is detected by nucleic acid sequencing analysis.

In certain non-limiting embodiments, the genomic is detected by probes present on a DNA array, chip, or a microarray. For example, and not by way of limitation, oligonucleotides corresponding to one or more genomic mutation can be immobilized on a chip which is then hybridized with labeled nucleic acids of a sample obtained from a subject. Positive hybridization signal is obtained with the sample containing the transcripts of the gene harboring a mutation.

In certain non-limiting embodiments, the genomic mutation is detected by a method comprising Reverse Transcription Polymerase Chain Reaction (“RT-PCR”). In certain embodiments, the mutated gene is detected by a method comprising RT-PCR using the one or more pairs of primers disclosed herein (see, for example, Table 1). In certain non-limiting embodiments, the genomic mutation is detected by antibody binding analysis such as, but not limited to, Western Blot analysis and immunohistochemistry.

TABLE 1
Primer sequences for mutant gene detection

| Gene | Exemplary Sequences |
|---|---|
| SLC45A2- | TTGATGTCTGCTCCCATCAGG (SEQ ID NO: 14) |
| AMACR | |
| SLC45A2- | CAGCTGGAGTTTCTCCATGAC (SEQ ID NO: 15) |
| AMACR | |
| SLC45A2- | GCCAGGAAAATGGGAGCTCTC (SEQ ID NO: 16) |
| AMACR | |
| SLC45A2- | TCCCTATCAGCTCATGAGCTC (SEQ ID NO: 17) |
| AMACR | |

### 5.4 Cancer Targets

In certain embodiments, the target of treatment is a cell that carries a mutation in the CTNNB1 gene, also known as catenin beta-1, or beta-catenin (β-catenin). In certain embodiment, the target of treatment is a cell that carries a thymidine to cytosine mutation at the position of 133 of the coding sequence of CTNNB1 (C. 133T>C). In certain embodiment, the target of treatment is a cell harboring a CTNNB1 gene mutation that converts the serine at position 45 of CTNNB1 to proline (CTNNB1^{S45P}). In certain embodiments, the target of treatment is a cell that carries a mutation in the SLTM gene, also known as SAFB-like transcription modulator. In certain embodiment, the target of treatment is a cell that carries a thymidine to guanine mutation at the position of 704 of the coding sequence of SLTM (C. 704 T>G, Hg19-Chr15: 58899823). In certain embodiment, the target of treatment is a cell harboring a SLTM gene mutation that converts the valine at position 235 SLMT to glycine (CTNNB1^{S45P}). In certain embodiment, the target of treatment is a cell that carries an adenine to guanine mutation at the position of 697 of the coding sequence of SLTM (C. 697A>G, HG19-Chr15: 58899830). In certain embodiment, the target of treatment is a cell that carries a cytosine to adenine mutation at the position of 694 of the coding sequence of SLTM (C. 694C>A, Hg19-Chr15: 58899833). In certain embodiments, the target of treatment is a cell that carries at least one fusion gene, e.g., SLC45A2-AMACR.

In certain embodiments, the target of treatment is a cancer cell that carries one of the presently disclosed genomic mutations. As one skilled in the art would appreciate, any cancer cells that carries one of the one of the presently disclosed genomic mutations can be targeted by the presently disclosed genome editing systems and methods.

Non-limiting examples of cancers that may be subject to the presently disclosed subject matter include cancers of the liver. The term “liver cancer” means malignancy of the liver, either a primary cancer or metastasized cancer. In certain embodiments, liver cancer includes, but is not limited to, cancer arising from hepatocytes, such as, for example, hepatomas, hepatocellular carcinomas (HCCs), fibrolamellar carcinoma, cholangiocarcinomas (or bile duct cancer), hepatoblastoma, hepatic carcinoma, hepatic angiosarcoma, or metastatic liver cancer.

In certain embodiments, the cancer is early-stage liver cancer. In certain embodiments, the cancer is late-stage liver cancer. In certain embodiments, metastatic liver cancer may have spread to secondary sites, including the lung, lymph nodes, bone, adrenal glands, peritoneum and or ometum, rectum, spleen, diaphragm, duodenum, esophagus, pancreas, seminal vesicle, and bladder, among others.

The term “metastasis” means the process by which cancer spreads from the place at which it first arose as a primary tumor to other locations in the body. The metastatic progression of a primary tumor reflects multiple stages, including dissociation from neighboring primary tumor cells, survival in the circulation, and growth in a secondary location.

The term “carcinoma” is art recognized and refers to malignancies of epithelial or endocrine tissues. The term includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “hepatocellular carcinoma” (HCC) refers to cancer that arises from hepatocytes, the major cell type of the liver. HCC is often associated with underlying liver diseases such as cirrhosis caused by chronic alcohol consumption, viral hepatitis, or other factors that lead to liver damage.

In certain embodiments, methods are provided for preventing liver cancer in an individual at risk of having liver cancer. In some embodiments, the individual has a liver condition selected from the group consisting of hepatitis B or C, cirrhosis of the liver, non-viral/non-alcoholic steatohepatitis, benign liver tumors, hemangiomas, hepatic adenomas, and focal nodular hyperplasia. Individuals considered at risk for developing cancer may benefit particularly from the present disclosure, primarily because prophylactic treatment can begin before there is any evidence of a tumor. Individuals “at risk” include, e.g., individuals exposed to carcinogens, e.g., by consumption, e.g., by inhalation and/or ingestion, at levels that have been shown statistically to promote cancer in susceptible individuals. Also included are individuals exposed to a virus, e.g., a hepatitis virus, e.g., hepatitis B virus (HBV). Also included are individuals at risk due to exposure to ultraviolet radiation, or their environment, occupation, and/or heredity, as well as those who show signs of a precancerous condition. Similarly, individuals in very early stages of cancer or development of metastases (i.e., only one or a few aberrant cells are present in the individual's body or at a particular site in an individual's tissue)) may benefit from such prophylactic treatment.

### 5.5 Methods of Treatment

The present disclosure provides methods of treating a subject carrying a gene mutation. In certain embodiments, the gene mutation can be a single nucleotide mutation. In certain embodiments, the gene mutation can be a gene fusion. In certain embodiments, the subject has, or is suspected of having, cancer or a neoplastic or pre-malignant condition that carries a gene mutation (a pre-malignant condition is characterized, inter alia, by the presence of pre-malignant or neoplastic cells). Non-limiting examples of a gene mutation are disclosed herein and in section 5.2. In certain embodiments, the methods of treatment include performing a targeted genome editing technique on one or more cancer cells within the subject to produce an anti-cancer or anti-neoplastic or anti-proliferative effect. Non-limiting examples of cancers that can be treated using the disclosed methods are provided in section 5.4. Non-limiting examples of genome editing techniques are disclosed in section 5.6.

An “anti-cancer/tumor effect” refers to one or more of a reduction in aggregate cancer cell mass, a reduction in cancer cell growth rate, a reduction in cancer progression, a reduction in cancer cell proliferation, a reduction in tumor mass, a reduction in tumor volume, a reduction in tumor cell proliferation, a reduction in tumor growth rate and/or a reduction in tumor metastasis. In certain embodiments, an anti-cancer effect can refer to a complete response, a partial response, a stable disease (without progression or relapse), a response with a later relapse or progression-free survival in a patient diagnosed with cancer. In certain embodiments, an anti-cancer effect can refer to the induction of cell death, e.g., in one or more cells of the cancer, and/or the increase in cell death within a tumor mass. Similarly, an “anti-neoplastic effect” refers to one or more of a reduction in aggregate neoplastic cell mass, a reduction in neoplastic cell growth rate, a reduction in neoplasm progression (e.g., progressive de-differentiation or epithelial to mesenchymal transition), a reduction in neoplastic cell proliferation, a reduction in neoplasm mass, a reduction in neoplasm volume, and/or a reduction in neoplasm growth rate.

In certain embodiments, a method of treating a subject comprises determining the presence of a gene mutation in a sample from the subject, where if the gene mutation is present in the sample, then performing a targeted genome editing technique on one or more cells within the subject. In certain embodiments, the genome editing technique results in the reduction and/or elimination of the expression of a gene mutation and/or the expression of the protein encoded by the gene mutation in one or more cells of the subject. In certain embodiments, the genome editing technique specifically targets the cells that carry gene mutation, e.g., by specifically targeting a nucleic acid sequence of the gene mutation. For example, and not by way of limitation, the methods of the current disclosure specifically target a single nucleotide mutation. In another example, and not by way of limitation, the methods of the current disclosure specifically target a gene fusion. In certain embodiments, the methods of the current disclosure involve the targeting of sequences that flank the gene mutation. In certain embodiments, the methods of the current disclosure involve the targeting of sequences that flank and partially overlap the gene mutation. Non-limiting examples of techniques for identifying and/or detecting a gene mutation are disclosed in section 5.3.

In certain embodiments, a method of treating a cancer in a subject comprises determining the presence of a gene mutation in a cancer cell-containing sample from the subject, where if the gene mutation is present in the sample then performing a targeted genome editing technique on one or more cancer cells within the subject to produce an anti-cancer effect or anti-neoplastic effect.

In certain embodiments, the gene mutation can be a single nucleotide mutation. In certain embodiments, the gene mutation can be a mutation of the CTNNB1 gene. In certain embodiments, the gene mutation can be a single nucleotide mutation of the CTNNB1 gene. In certain embodiments, the gene mutation can be a S45P mutation of CTNNB1.

In certain embodiments, the gene mutation can be a mutation of the SLTM gene. In certain embodiments, the gene mutation can be a single nucleotide mutation of the SLTM gene.

In certain embodiments, the gene mutation can be a V235G mutation of the SLTM gene.

In certain embodiments, the gene mutation can be a gene fusion. In certain embodiments, the gene mutation can be a gene fusion. In certain embodiments, the gene mutation can be a SLC45A2-AMACR gene fusion.

In certain embodiments, the method can include determining the presence or absence of a gene mutation. For example, and not by way of limitation, the method can include determining the presence or absence of one or more gene mutations disclosed herein.

In certain embodiments, the method of treating a subject comprises determining the presence of one or more gene mutations in a sample of the subject, where if one or more gene mutations are detected in the sample then performing a targeted genome editing technique on the one or more gene mutations in one or more cancer cells within the subject to produce an anti-cancer effect.

In certain embodiments, the method of treating a subject having a cancer comprises determining the presence, in one or more cancer cell(s) of the subject, of one or more gene mutations, where if one or more gene mutations are detected in the cancer cell(s) then performing a genome editing technique targeting the gene mutation present within one or more cancer cells of the subject to produce an anti-cancer effect. In certain embodiments, the normal or non-cancerous cells that are adjacent to the cancer are not subjected to a genome editing technique as the gRNAs are specific for the sequences of the fusion gene, e.g., specific to the sequence of the breakpoint.

In certain embodiments, the method of treating a subject having a cancer comprises determining the presence, in one or more cancer cell(s) of the subject, of one or more gene mutations, where if one or more gene mutations are detected in the cancer cell(s) then performing a targeted genome editing technique on one or more cancer cells within the subject to produce an anti-cancer effect.

In certain embodiments, the method of treating a subject comprises determining the presence, in one or more cell(s) of the subject, of one or more gene mutations, where if one or more gene mutations are detected in the cell(s) then performing a targeted genome editing technique on one or more cells within the subject, e.g., to reduce and/or eliminate the expression of the gene mutation and/or reduce and/or eliminate the expression of the protein encoded by the fusion gene in the one or more cells of the subject.

In certain embodiments, the present disclosure provides a method of producing an anti-cancer effect in a subject having a cancer comprising performing a targeted genome editing technique on one or more cancer cells that contain one or more gene mutations within the subject, e.g., by targeting the one or more gene mutations, to produce an anti-cancer effect.

The present disclosure further provides a method of preventing, minimizing and/or reducing the growth of a tumor comprising determining the presence of one or more gene mutations in a sample of the subject, where if one or more gene mutations are present in the sample then performing a genome editing technique targeting the gene mutation present within the tumor of the subject to prevent, minimize and/or reduce the growth of the tumor.

The present disclosure provides a method of preventing, minimizing and/or reducing the growth and/or proliferation of a cancer cell comprising determining the presence of one or more gene mutations in a sample of the subject, where if one or more gene mutations are present in the sample then performing a genome editing technique targeting the one or more gene mutations present within the cancer cell of the subject to prevent, minimize and/or reduce the growth and/or proliferation of the cancer cell. In certain embodiments, the sequences that flank the one or more gene mutations can be targeted by the genome editing technique.

In certain non-limiting embodiments, the present disclosure provides for methods of treating and/or inhibiting the progression of cancer and/or tumor and/or neoplastic growth in a subject comprising determining the presence of one or more gene mutation in a sample of the subject, where if one or more gene mutations are present in the sample then performing a targeted genome editing technique on one or more cells from the cancer and/or tumor of the subject to treat and/or inhibit the progression of the cancer and/or the tumor.

In certain embodiments, the present disclosure provides a method for lengthening the period of survival of a subject having a cancer. In certain embodiments, the method comprises determining the presence of one or more gene mutations in a sample of the subject, where if one or more gene mutations are present in the sample then performing a targeted genome editing technique on one or more cancer cells within the subject to produce an anti-cancer effect.

In certain embodiments, the period of survival of a subject having cancer can be lengthened by about 1 month, about 2 months, about 4 months, about 6 months, about 8 months, about 10 months, about 12 months, about 14 months, about 18 months, about 20 months, about 2 years, about 3 years, about 5 years or more using the disclosed methods.

In certain embodiments, the present disclosure provides an agent, or a composition comprising an agent, capable of targeted genome editing for use in a method to treat a subject. For example, and not by way of limitation, the present disclosure provides an agent capable of targeted genome editing for use in a method to treat or prevent cancer in a subject, wherein the method comprises performing a targeted genome editing procedure using the agent on one or more cells, e.g., cancer cells, that contain a gene mutation within the subject. In certain embodiments, the disclosure provides an agent, or a composition thereof, capable of targeted genome editing for use in a method to treat or prevent cancer in a subject, wherein the method comprises (i) determining the presence of a gene mutation in a cancer sample of the subject and (ii) where the sample contains a gene mutation, performing a targeted genome editing procedure using the agent on one or more cancer cells within the subject. In certain embodiments, the agent targets a single nucleotide mutation or a specific chromosomal breakpoint of the fusion gene. In certain embodiments, the methods of the current disclosure involve the targeting of sequences that flank the gene mutation. In certain embodiments, the agent is an endonuclease. For example, and not by way of limitation, the endonuclease is a Cas9 protein. In certain embodiments, the endonuclease is a mutated form of Cas9, e.g., Cas9^{D10A}. In certain embodiments, the agent is an endonuclease, e.g., Cas9, in complex with one or more gRNAs (e.g., a ribonucleoprotein). In certain embodiments, the agent is an siRNA molecule.

In certain embodiments, the present disclosure provides a method of determining a treatment for a subject having one or more cells that contains one or more gene mutations. In certain embodiments, the method can include i) providing a sample from the subject; ii) determining whether one or more cells of the subject contains one or more gene mutations; and iii) instructing a genome editing technique to be performed if one or more gene mutations are detected in the one or more cells, wherein the genome editing technique targets the one or more of the gene mutations detected in the one or more cells In certain embodiments, the genome editing technique is performed using the CRISPR/Cas9 system.

In certain embodiments, the sample in which the one or more gene mutations are detected is liver cancer.

In certain embodiments, the sample in which the one or more gene mutations are detected is a hepatocellular carcinoma.

In certain embodiments, the gene mutation in a sample is detected by genome sequencing. In certain embodiments, the gene mutation in a sample is detected by RNA sequencing. For example, and not by way of limitation, RNA sequencing can be performed using the primers disclosed in Table 1. In certain embodiments, the fusion gene in a sample is detected by FISH.

In certain embodiments, the methods of treating a subject, e.g., a subject that has a cancer that carries a gene mutation disclosed herein, can further comprise administering a therapeutically effective amount of an anti-cancer agent or agent that results in an anti-neoplastic effect. A “therapeutically effective amount” refers to an amount that is able to achieve one or more of the following: an anti-cancer effect, an anti-neoplastic effect, a prolongation of survival and/or prolongation of period until relapse. An anti-cancer agent can be any molecule, compound chemical or composition that has an anti-cancer effect. Anti-cancer agents include, but are not limited to, chemotherapeutic agents, radiotherapeutic agents, cytokines, anti-angiogenic agents, apoptosis-inducing agents or anti-cancer immunotoxins. In certain non-limiting embodiments, a genome-editing technique, disclosed herein, can be used in combination with one or more anti-cancer agents. “In combination with,” as used herein, means that the genome-editing technique and the one or more anti-cancer agents (or agents that are that results in an anti-neoplastic effect) are part of a treatment regimen or plan for a subject.

### 5.6 Genome Targeting/Editing Techniques

Genome editing is a technique in which endogenous chromosomal sequences present in one or more cells within a subject, can be edited, e.g., modified, using targeted endonucleases and single-stranded nucleic acids. The genome editing method can result in the insertion of a nucleic acid sequence at a specific region within the genome, the excision of a specific sequence from the genome and/or the replacement of a specific genomic sequence with a new nucleic acid sequence. In certain embodiments, the genome editing technique can results in the repression of the expression of a gene, e.g., a mutant gene or a fusion gene. For example, and not by way of limitation, a nucleic acid sequence can be inserted in a gene harboring a mutation or at a chromosomal breakpoint of a fusion gene.

In certain embodiments, the nuclease is selected from a zinc finger nuclease, a meganuclease, a TALE nuclease, or a CRISPR system nuclease. In certain embodiments, the nuclease is a CRISPR system nuclease.

A non-limiting example of a genome editing technique for use in the disclosed methods is the CRISPR system, e.g., CRISPR Cas 9 system. Non-limiting examples of such genome editing techniques are disclosed in PCT Application Nos. WO 2014/093701 and WO 2014/165825, the contents of which are hereby incorporated by reference in their entireties.

In certain embodiments, the genome editing technique can include the use of one or more guide RNAs (gRNAs), complementary to a specific sequence within a genome, e.g., a ga mutation site or a chromosomal breakpoint associated with a fusion gene, including protospacer adjacent motifs (PAMs), to guide a nuclease, e.g., an endonuclease, to the specific genomic sequence. In certain embodiments, the genome editing technique can include the use of one or more guide RNAs (gRNAs), complementary to the sequences that are adjacent to and/or overlap the mutation site or chromosomal breakpoint (see, e.g., FIGS. 1, 2 and 5), to guide one or more nucleases.

In certain embodiments, the one or more gRNAs can include a targeting sequence that is complementary to a sequence present within the gene harboring a mutation, e.g., complementary to the sequences that are adjacent to and/or overlap the mutation site. In certain embodiments, the one or more gRNAs used for targeting the gene harboring a mutation can comprise a sequence that is at least partially complementary to the mutation sequence of the gene harboring a mutation and at least partially complementary to a non-mutated sequence of the gene harboring a mutation. In certain embodiments, the one or more gRNAs can include a targeting sequence that is complementary to a sequence present within the fusion gene, e.g., complementary to the sequences that are adjacent to and/or overlap the chromosomal breakpoint. In certain embodiments, the one or more gRNAs used for targeting the fusion gene can comprise a sequence that is at least partially complementary to the breakpoint sequence of the fusion gene and at least partially complementary to a sequence of one of the genes that comprises the fusion gene. In certain embodiments, the targeting sequences are about 10 to about 50 nucleotides in length, e.g., from about 10 to about 45 nucleotides, from about 10 to about 40 nucleotides, from about 10 to about 35 nucleotides, from about 10 to about 30 nucleotides, from about 10 to about 25 nucleotides, from about 10 to about 20 nucleotides, from about 10 to about 15 nucleotides, from about 15 to about 50 nucleotides, from about 20 to about 50 nucleotides, from about 25 to about 50 nucleotides, from about 30 to about 50 nucleotides, from about 35 to about 50 nucleotides, from about 40 to about 50 nucleotides or from about 45 to about 50 nucleotides in length. In certain embodiments, the targeting sequence is greater than about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides in length.

In certain embodiments, the one or more gRNAs comprise a pair of offset gRNAs complementary to opposite strands of the target site. In certain embodiments, the one or more gRNAs comprises a pair of offset gRNAs complementary to opposite strands of the target site to generate offset nicks by an endonuclease. In certain embodiments, the offset nicks are induced using a pair of offset gRNAs with a nickase, e.g., a Cas9 nickase such as Cas9^{D10A} In certain embodiments, the pair of offset gRNAs are offset by at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 88, 89, 90, 91, 92,93, 94, 95,96, 97, 98, 99, or at least 100 nucleotides. In certain embodiments, the pair of offset sgRNAs are offset by about 5 to about 100 nucleotides, about 10 to about 50 nucleotides, about 10 to about 40 nucleotides, about 10 to about 30 nucleotides, about 10 to about 20 nucleotides or about 15 to 30 nucleotides.

In certain non-limiting embodiments, a PAM can be recognized by a CRISPR endonuclease such as a Cas protein. Non-limiting examples of Cas proteins include, but are not limited to, Cas1, Cas1B, Cas2, Cas3, Cas4, Cas5, Cas6, Cas7, Cas8, Cas9 (also known as Csn1 or Csx12), Cas1O, Csy1, Csy2, Csy3, Cse 1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, Cas9HiFi, homologues thereof or modified versions thereof.

In certain embodiments, the endonuclease can be the clustered, regularly interspaced short palindromic repeat (CRISPR) associated protein 9 (Cas9) endonuclease. In certain embodiments, the Cas9 endonuclease is obtained from Streptococcus pyogenes. In certain embodiments, the Cas9 endonuclease is obtained from Staphylococcus aureus. In certain embodiments, the endonuclease can result in the cleavage of the targeted genome sequence and allow modification of the genome at the cleavage site through nonhomologous end joining (NHEJ) or homologous recombination. In certain embodiments, the Cas9 endonuclease can be a mutated form of Cas9, e.g., that generates a single-strand break or “nick.” For example, and not by way of limitation, the Cas9 protein can include the D10A mutation, i.e., Cas9^{D10A} (see Cong et al. Science. 339:819-823 (2013); Gasiunas et al. PNAS 109:E2579-2586 (2012); and Jinek et al. Science. 337:816-821 (2012), the contents of which are incorporated by reference herein).

In certain embodiments, the genome editing method and/or technique can be used to target one or more sequences of a gene or fusion gene present in a cell, e.g., in a cancer cell, to promote homologous recombination to insert a nucleic acid into the genome of the cell. For example, and not by way of limitation, the genome editing technique can be used to target the region of mutation site or where two genes of a fusion gene are joined together (i.e., the junction and/or chromosomal breakpoint).

In certain embodiments, the genome editing method and/or technique can be used to target one or more sequences of a gene or fusion gene present in a cell, e.g., in a cancer cell, to promote homologous recombination to insert a nucleic acid encoding a suicide gene into the genome of the cell. For example, and not by way of limitation, the genome editing technique can be used to target the region of mutation site or where two genes of a fusion gene are joined together (i.e., the junction and/or chromosomal breakpoint). In certain embodiments, the suicide gene is selected from herpes simplex virus thymidine kinase (HSV-tk), Exotoxin A from Pseudomonas aeruginosa, Diphtheria toxin from Corynebacterium Diphtheriai, Ricin or abrin from Ricinus communi (castor oil plant), Cytosine deaminase, Carboxyl esterase, Varicella Zoster virus (VZV) thymidine kinase, or a combination thereof. In certain embodiments, the suicide gene is herpes simplex virus thymidine kinase (HSV-tk). In certain embodiments, the nucleic acid encoding the suicide gene comprises a first homology recombination arm and a second homology recombination arm. In certain embodiments, the first homology recombination arm and the second homology recombination arm flank the nucleic acid encoding the suicide gene.

In certain embodiments, the genome editing method and/or technique can be used to knockout the gene or fusion gene, e.g., by excising out at least a portion of the gene or fusion gene, to disrupt the gene or fusion gene sequence. For example, and not by way of limitation, an endonuclease, e.g., a wild type Cas9 endonuclease, can be used to specifically cleave the double-stranded DNA sequence of a gene or fusion gene, and in the absence of a homologous repair template non-homologous end joining can result in indels to disrupt the gene or fusion gene sequence.

In certain embodiments, the genome editing method and/or technique can be used to repress the expression of the gene or fusion gene, e.g., by using a nuclease-deficient Cas9. For example, and not by way of limitation, mutations in a catalytic domain of Cas9, e.g., H840A in the HNH domain and D10A in the RuvC domain, inactivates the cleavage activity of Cas9 but do not prevent DNA binding. In certain embodiments, Cas9^{D10A H840A} (referred to herein as dCas9) can be used to target the region of a mutation site or where two genes of a fusion gene are joined together without cleavage, and by fusing with various effector domains, dCas9 can be used to silence the gene or fusion gene.

As normal, non-cancerous cells do not contain the genomic mutation or fusion gene, cells can be specifically targeted using this genome editing technique. In certain embodiments, the genome editing technique can be used to target a gene harboring a mutation, including, but not limited to, CTNNB1 and SLTM. In certain embodiments, the genome editing technique can be used to target the junction (i.e., breakpoint) of a fusion gene including, but not limited to, SLC45A2-AMACR.

In certain embodiments, the one or more gRNAs that can be used in the disclosed methods can target sequences harboring a genomic mutation or a fusion gene chromosomal breakpoint as disclosed herein and within the FIGS. 1A, 3A, and 5A. In certain embodiments, the one or more gRNAs used in the disclosed methods can be about 80%, about 85%, about 90%, about 91%, about 92%, about 93%, about 94%, about 95%, about 96%, about 97%, about 98% or about 99% homologous and/or complementary to the sequences harboring the genomic mutation or the fusion gene chromosomal breakpoint disclosed herein.

In certain embodiments, the gRNAs can be designed to target (e.g., be complementary to) the sequences flanking the mutation sites or chromosomal breakpoint region (see, for example, FIGS. 1, 3 and 5) to guide an endonuclease, e.g., Cas9^{D10A}, to the chromosomal breakpoint region or a region surrounding the breakpoint. Non-limiting examples of the sequences of the gRNAs that can be used in the disclosed methods are detailed in FIGS. 1A, 3A, 5A (e.g., SEQ ID nOs: 7-13). In certain embodiments, the one or more gRNAs used in the disclosed methods can be about 80%, about 85%, about 90%, about 91%, about 92%, about 93%, about 94%, about 95%, about 96%, about 97%, about 98% or about 99% homologous to the gRNAs disclosed herein. In certain embodiments, the disclosed gRNAs can include about 1, about 2, about 3, about 4 or about 5 nucleotide substitutions and/or mutations.

In certain embodiments, the one or more gRNAs targeting CTNNB1 flank the mutation site of the CTNNB1 gene. In certain embodiments, one or more gRNAs used to target CTNNB1 gene can have a nucleotide sequence that comprises one or more of the nucleotide sequences set forth in FIG. 1A. In certain embodiments, the one or more gRNAs for targeting CTNNB1 can be about 80%, about 85%, about 90%, about 91%, about 92%, about 93%, about 94%, about 95%, about 96%, about 97%, about 98% or about 99% homologous to the gRNAs disclosed in FIG. 1A.

In certain embodiments, the gene is CTNNB1.

In certain embodiments, the one or more gRNAs targeting SLTM flank the mutation site of the SLTM gene. In certain embodiments, one or more gRNAs used to target SLTM gene can have a nucleotide sequence that comprises one or more of the nucleotide sequences set forth in FIG. 5A. In certain embodiments, the one or more gRNAs for targeting SLTM can be about 80%, about 85%, about 90%, about 91%, about 92%, about 93%, about 94%, about 95%, about 96%, about 97%, about 98% or about 99% homologous to the gRNAs disclosed in FIG. 5A.

In certain embodiments, the gene is SLTM.

In certain embodiments, the one or more gRNAs can target intron 2 of SLC45A2 and intron 1 of AMACR, e.g., one gRNA can target intron 2 of SLC45A2 and the second gRNA can target intron 1 of AMACR, which flank the breakpoint of the SLC45A2-AMACR fusion gene. In certain embodiments, one or more gRNAs used to target SLC45A2-AMACR fusion gene can have a nucleotide sequence that comprises one or more of the nucleotide sequences set forth in FIG. 3A. In certain embodiments, the one or more gRNAs for targeting SLC45A2-AMACR can be about 80%, about 85%, about 90%, about 91%, about 92%, about 93%, about 94%, about 95%, about 96%, about 97%, about 98% or about 99% homologous to the gRNAs disclosed in FIG. 3A.

In certain embodiments, the fusion gene is SLC45A2-AMACR.

In certain embodiments, the disclosed genome editing technique can be used to promote homologous recombination with a sequence of a gene or fusion gene, e.g., at a mutation site or at a chromosomal breakpoint (junction) of a fusion gene, in one or more cells of a subject to allow the insertion of a nucleic acid sequence of a suicide gene that when expressed results in or can lead to the death, e.g., apoptosis, of the one or more cells.

For example, and not by way of limitation, the nucleic acid sequence (also referred to herein as a donor nucleic acid) can encode a suicide gene selected from Herpes Simplex Virus 1 (HSV-1) thymidine kinase, Exotoxin A from Pseudomonas aeruginosa, Diphtheria toxin from Corynebacterium Diphtheriai, Ricin or abrin from Ricinus communi (castor oil plant), Cytosine deaminase from bacteria or yeast, Carboxyl esterase or Varicella Zoster virus (VZV) thymidine kinase. Additional non-limiting examples of nucleic acids and/or genes that can be inserted into the genome of a cell carrying a genomic mutation or a fusion gene to induce cell death are disclosed in Rajab et al. (2013) (J. of Genetics Syndromes and Gene Therapy, 4(9):187) and Zarogoulidis et al. (2013) (J. of Genetics Syndromes and Gene Therapy, 4(9):pii: 16849). In certain non-limiting embodiments, the nucleic acid sequence, e.g., the HSV-1 thymidine kinase nucleic acid sequence, is not operably linked to a regulatory sequence promoter (e.g., a promoter) and requires integration into the genome for expression. For example, and not by way of limitation, the promoter of the gene or head gene of the fusion gene can promote the expression of the donor nucleic acid sequence.

In certain embodiments, the suicide gene is herpes simplex virus thymidine kinase (HSV-tk). In certain embodiments, HSV-tk comprises an amino acid sequence at least about 80%, about 85%, about 90%, about 91%, about 92%, about 93%, about 94%, about 95%, about 96%, about 97%, about 98%, about 99%, or about 100% homologous to the amino acid sequence set forth in SEQ ID NO: 18. In certain embodiments, HSV-tk comprises an amino acid sequence at least about 80%, about 85%, about 90%, about 91%, about 92%, about 93%, about 94%, about 95%, about 96%, about 97%, about 98%, or about 99% identical to the amino acid sequence set forth in SEQ ID NO: 18. In certain embodiments, HSV-tk comprises the amino acid sequence set forth in SEQ ID NO: 18. In certain embodiments, HSV-tk consists of the amino acid sequence set forth in SEQ ID NO: 18. SEQ ID NO: 18 is provided below:

[SEQ ID NO: 18]
MASYPCHQHASAFDQAARSRGHSNRRTALRPRRQQEATEVRLEQKMPTL
LRVYIDGPHGMGKTTTTQLLVALGSRDDIVYVPEPMTYWQVLGASETIA
NIYTTQHRLDQGEISAGDAAVVMTSAQITMGMPYAVTDAVLAPHIGGEA
GSSHAPPPALTLIFDRHPIAALLCYPAARYLMGSMTPQAVLAFVALIPP
TLPGTNIVLGALPEDRHIDRLAKRQRPGERLDLAMLAAIRRVYGLLANT
VRYLQGGGSWREDWGQLSGTAVPPQGAEPQSNAGPRPHIGDTLFTLFRA
PELLAPNGDLYNVFAWALDVLAKRLRPMHVFILDYDQSPAGCRDALLQL
TSGMVQTHVTTPGSIPTICDLARTFAREMGEAN

In certain embodiments, HSV-tk comprises an amino acid sequence at least about 80%, about 85%, about 90%, about 91%, about 92%, about 93%, about 94%, about 95%, about 96%, about 97%, about 98%, about 99%, or about 100% homologous to the amino acid sequence set forth in SEQ ID NO: 19. In certain embodiments, HSV-tk comprises an amino acid sequence at least about 80%, about 85%, about 90%, about 91%, about 92%, about 93%, about 94%, about 95%, about 96%, about 97%, about 98%, or about 99% identical to the amino acid sequence set forth in SEQ ID NO: 19. In certain embodiments, HSV-tk comprises the amino acid sequence set forth in SEQ ID NO: 19. In certain embodiments, HSV-tk consists of the amino acid sequence set forth in SEQ ID NO: 19. SEQ ID NO: 19 is provided below:

[SEQ ID NO: 19]
MASHAGQQHAPAFGQAARASGPTDGRAASRPSHRQGASEARGDPELPTL
LRVYIDGPHGVGKTTTSAQLMEALGPRDNIVYVPEPMTYWQVLGASETL
TNIYNTQHRLDRGEISAGEAAVVMTSAQITMSTPYAATDAVLAPHIGGE
AVGPQAPPPALTLVFDRHPIASLLCYPAARYLMGSMTPQAVLAFVALMP
PTAPGTNLVLGVLPEAEHADRLARRQRPGERLDLAMLSAIRRVYDLLAN
TVRYLQRGGRWREDWGRLTGVAAATPRPDPEDGAGSLPRIEDTLFALFR
VPELLAPNGDLYHIFAWVLDVLADRLLPMHLFVLDYDQSPVGCRDALLR
LTAGMIPTRVTTAGSIAEIRDLARTFAREVGGV

In certain embodiments, HSV-tk comprises an amino acid sequence at least about 80%, about 85%, about 90%, about 91%, about 92%, about 93%, about 94%, about 95%, about 96%, about 97%, about 98%, about 99%, or about 100% homologous to the amino acid sequence set forth in SEQ ID NO: 20. In certain embodiments, HSV-tk comprises an amino acid sequence at least about 80%, about 85%, about 90%, about 91%, about 92%, about 93%, about 94%, about 95%, about 96%, about 97%, about 98%, or about 99% identical to the amino acid sequence set forth in SEQ ID NO: 20. In certain embodiments, HSV-tk comprises the amino acid sequence set forth in SEQ ID NO: 20. In certain embodiments, HSV-tk consists of the amino acid sequence set forth in SEQ ID NO: 20. SEQ ID NO: 20 is provided below:

[SEQ ID NO: 20]
MASYPCHQHASAFDQAARSRGHSNRRTALRPRRQQEATEVRLEQKMPTL
LRVYIDGPHGMGKTTTTQLLVALGSRDDIVYVPEPMTYWQVLGASETIA
NIYTTQHRLDQGEISAGDAAVVMTSAQITMGMPYAVTDAVLAPHVGGEA
GSSHAPPPALTLIFDRHPIAALLCYPAARYLMGSMTPQAVLAFVALIPP
TLPGTNIVLGALPEDRHIDRLAKRQRPGERLDLAMLAAIRRVYGLLANT
VRYLQGGGSWWEDWGQLSGTAVPPQGAEPQSNAGPRPHIGDTLFTLFRA
PELLAPNGDLYNVFAWALDVLAKRLRPMHVFILDYDQSPAGCRDALLQL
TSGMVQTHVTTPGSIPTICDLARTFAREMGEAN

In certain embodiments, HSV-tk comprises an amino acid sequence at least about 80%, about 85%, about 90%, about 91%, about 92%, about 93%, about 94%, about 95%, about 96%, about 97%, about 98%, about 99%, or about 100% homologous to the amino acid sequence set forth in SEQ ID NO: 21. In certain embodiments, HSV-tk comprises an amino acid sequence at least about 80%, about 85%, about 90%, about 91%, about 92%, about 93%, about 94%, about 95%, about 96%, about 97%, about 98%, or about 99% identical to the amino acid sequence set forth in SEQ ID NO: 21. In certain embodiments, HSV-tk comprises the amino acid sequence set forth in SEQ ID NO: 21. In certain embodiments, HSV-tk consists of the amino acid sequence set forth in SEQ ID NO: 21. SEQ ID NO: 21 is provided below:

[SEQ ID NO: 21]
MASYPCHQHASAFDQAARSRGHSNRRTALRPRRQQEATEVRLEQKMPTL
LRVYIDGPHGMGKTTTTQLLVALGSRDDIVYVPEPMTYWQVLGASETIA
NIYTTQHRLDQGEISAGDAAVVMTSAQITMGMPYAVTDAVLAPHIGGEA
GSSHAPPPALTLIFDRHPIAALLCYPAARYLMGSMTPQAVLAFVALIPP
TLPGTNIVLGALPEDRHIDRLAKRQRPGERLDLAMLAAIRRVYGLLANT
VRYLQGGGSWREDWGQLSGTAVPPQGAEPQSNAGPRPHIGDTLFTLFRA
PELLAPNGDLYNVFAWALDVLAKRLRPMHVFILDYDQSPAGCRDALLQL
TSGMVQTHVTTPGSIPTICDLARMFAREMGEAN

In certain embodiments, HSV-tk comprises an amino acid sequence at least about 80%, about 85%, about 90%, about 91%, about 92%, about 93%, about 94%, about 95%, about 96%, about 97%, about 98%, about 99%, or about 100% homologous to the amino acid sequence set forth in SEQ ID NO: 22. In certain embodiments, HSV-tk comprises an amino acid sequence at least about 80%, about 85%, about 90%, about 91%, about 92%, about 93%, about 94%, about 95%, about 96%, about 97%, about 98%, or about 99% identical to the amino acid sequence set forth in SEQ ID NO: 22. In certain embodiments, HSV-tk comprises the amino acid sequence set forth in SEQ ID NO: 22. In certain embodiments, HSV-tk consists of the amino acid sequence set forth in SEQ ID NO: 22. SEQ ID NO: 22 is provided below:

[SEQ ID NO: 22]
MASYPGHQHASAFDQAARSRGHSNRRTALRPRRQQEATEVRPEQKMPTL
LRVYIDGPHGMGKTTTTQLLVALGSRDDIVYVPEPMTYWRVLGASETIA
NIYTTQHRLDQGEISAGDAAVVMTSAQITMGMPYAVTDAVLAPHIGGEA
GSSHAPPPALTLIFDRHPIAALLCYPAARYLMGSMTPQAVLAFVALIPP
TLPGTNIVLGALPEDRHIDRLAKRQRPGERLDLAMLAAIRRVYGLLANT
VRYLQGGGSWREDWGQLSGTAVPPQGAEPQSNAGPRPHIGDTLFTLFRA
PELLAPNGDLYNVFAWALDVLAKRLRPMHVFILDYDQSPAGCRDALLQL
TSGMIQTHVTTPGSIPTICDLARTFAREMGEAN

In certain embodiments, HSV-tk comprises an amino acid sequence at least about 80%, about 85%, about 90%, about 91%, about 92%, about 93%, about 94%, about 95%, about 96%, about 97%, about 98%, about 99%, or about 100% homologous to the amino acid sequence set forth in SEQ ID NO: 23. In certain embodiments, HSV-tk comprises an amino acid sequence at least about 80%, about 85%, about 90%, about 91%, about 92%, about 93%, about 94%, about 95%, about 96%, about 97%, about 98%, or about 99% identical to the amino acid sequence set forth in SEQ ID NO: 23. In certain embodiments, HSV-tk comprises the amino acid sequence set forth in SEQ ID NO: 23. In certain embodiments, HSV-tk consists of the amino acid sequence set forth in SEQ ID NO: 23. SEQ ID NO: 23 is provided below:

[SEQ ID NO: 23]
MASYPCHQHASAFDQAARSRGHSNRRTALRPRRQQEATEVRLEQKMPTL
LRVYIDGPHGMGKTTTTQLLVALGSRDDIVYVPEPMTYWQVLGASETIA
NIYTTQHRLDQGEISAGDAAVVMTSAQITMGMPYAVTDAVLAPHIGGEA
GSSHAPPPALTLIFDRHPIAALLCYPAARYLMGSMTPQAVLAFVALIPP
TLPGTNIVLGALPEDRHIDRLAKRQRPGERLDLAMLAAIRRVYGLLANT
VRYLQGGGSWREDWGQLSGTAVPPQGAEPQSNAGPRPHIGDTLFTLFRA
PELLAPNGDLYNVFAWALDVLAKRLRPMHVFILDYDQSPAGCRDALLQL
TSGMVQTHVTTPGSIPTICDLARMFAREMGEAN

In certain embodiments, HSV-tk comprises an amino acid sequence at least about 80%, about 85%, about 90%, about 91%, about 92%, about 93%, about 94%, about 95%, about 96%, about 97%, about 98%, about 99%, or about 100% homologous to the amino acid sequence set forth in SEQ ID NO: 24. In certain embodiments, HSV-tk comprises an amino acid sequence at least about 80%, about 85%, about 90%, about 91%, about 92%, about 93%, about 94%, about 95%, about 96%, about 97%, about 98%, or about 99% identical to the amino acid sequence set forth in SEQ ID NO: 24. In certain embodiments, HSV-tk comprises the amino acid sequence set forth in SEQ ID NO: 24. In certain embodiments, HSV-tk consists of the amino acid sequence set forth in SEQ ID NO: 24. SEQ ID NO: 24 is provided below:

[SEQ ID NO: 24]
MASHAGQQHAPAFGQAARASGPTDGRAASRPSHRQGASGARGDPELPTL
LRVYIDGPHGVGKTTTSAQLMEALGPRDNIVYVPEPMTYWQVLGASETL
TNIYNTQHRLDRGEISAGEAAVVMTSAQITMSTPYAATDAVLAPHIGGE
AVGPQAPPPALTLVFDRHPIASLLCYPAARYLMGSMTPQAVLAFVALMP
PTAPGTNLVLGVLPEAEHADRLARRQRPGERLDLAMLSAIRRVYDLLAN
TVRYLQRGGRWREDWGRLTGVAAATPRPDPEDGAGSLPRIEDTLFALFR
VPELLAPNGDLYHIFAWVLDVLADRLLPMHLFVLDYDQSPVGCRDALLR
LTAGMIPTRVTTAGSIAEIRDLARTFAREVGGV

In certain embodiments, the suicide gene is herpes simplex virus thymidine kinase (HSV-tk). In certain embodiments, HSV-tk comprises an amino acid sequence at least about 80%, about 85%, about 90%, about 91%, about 92%, about 93%, about 94%, about 95%, about 96%, about 97%, about 98%, about 99%, or about 100% homologous to the amino acid sequence set forth in SEQ ID NO: 25. In certain embodiments, HSV-tk comprises an amino acid sequence at least about 80%, about 85%, about 90%, about 91%, about 92%, about 93%, about 94%, about 95%, about 96%, about 97%, about 98%, or about 99% identical to the amino acid sequence set forth in SEQ ID NO: 25. In certain embodiments, HSV-tk comprises the amino acid sequence set forth in SEQ ID NO: 25. In certain embodiments, HSV-tk consists of the amino acid sequence set forth in SEQ ID NO: 25. SEQ ID NO: 25 is provided below:

[SEQ ID NO: 25]
MASYPGHQHASAFDQAARSRGHSNRRTALRPRRQQEATEVRPEQKMPTL
LRVYIDGPHGMGKTTTTQLLVALGSRDDIVYVPEPMTYWRVLGASETIA
NIYTTQHRLDQGEISAGDAAVVMTSAQITMGMPYAVTDAVLAPHIGGEA
GSSHAPPPALTLIFDRHPIAALLCYPAARYLMGSMTPQAVLAFVALIPP
TLPGTNIVLGALPEDRHIDRLAKRQRPGERLDLAMLAAIRRVYGLLANT
VRYLQCGGSWREDWGQLSGTAVPPQGAEPQSNAGPRPHIGDTLFTLFRA
PELLAPNGDLYNVFAWALDVLAKRLRSMHVFILDYDQSPAGCRDALLQL
TSGMVQTHVTTPGSIPTICDLARTFAREMGEAN

In certain embodiments where a nucleic acid encoding HSV-1 thymidine kinase is inserted in the genome of one or more cells of a subject, the cell is contacted with the guanine derivative, ganciclovir, or its oral homolog, valganciclovir. In certain embodiments where a nucleic acid encoding HSV-1 thymidine kinase is inserted in the genome of one or more cells of a subject, a therapeutically effective amount of the guanine derivative, ganciclovir, or its oral homolog, valganciclovir, can be administered to the subject.

As illustrated in FIGS. 6A-6C, the presently disclosed genome editing systems and method can be used to deliver a suicide gene (e.g., HSV-tk) and nucleases in order to target a genomic mutation of interest (e.g., one described in Section 5.2). The cells are then contacted with ganciclovir and/or valganciclovir. HSV-1 thymidine kinase can phosphorylate and convert ganciclovir and/or valganciclovir into the triphosphate forms of ganciclovir and/or valganciclovir in the one or more cells of the subject. The triphosphate form of ganciclovir and/or valganciclovir acts as competitive inhibitor of deoxyguanosine triphosphate (dGTP) and is a poor substrate of DNA elongation and can result in the inhibition of DNA synthesis. The inhibition of DNA synthesis, in turn, can result in the reduction and/or inhibition of growth and/or survival and/or cell death of cancer cells that contain a genomic mutation or a targeted chromosomal breakpoint and the integrated HSV-1 thymidine kinase nucleic acid sequence. Thus, this genome editing method can be used to produce an anti-cancer effect in a subject that has been determined to have a genomic mutation or a fusion gene.

In certain embodiments, a genome editing technique of the present disclosure can include the introduction of an expression vector comprising a nucleic acid sequence that encodes a Cas protein or a mutant thereof, e.g., Cas9^{D10A, into one or more cells of the subject, e.g., cancer cells, carrying a gene or fusion gene. In certain embodiments, the cells are not prostate cancer cells. In certain embodiments, the vector can further comprise one or more gRNAs for targeting the Cas}9 protein to a specific nucleic acid sequence within the genome.

In certain embodiments, the expression vector can be a viral vector.

In certain embodiments, the one or more gRNAs can hybridize to a target sequence within a gene or fusion gene. For example, and not by way of limitation, the one or more gRNAs can target the mutation site of a gene and/or target the one or more sequences that flank the mutation site region. In another example, and not by way of limitation, the one or more gRNAs can target the chromosomal breakpoint of a fusion gene and/or target the one or more sequences that flank the chromosomal breakpoint region. Non-limiting examples of sequences of genomic mutation sites or fusion gene chromosomal breakpoints are disclosed herein and within the FIGS. 1A, 3A, and 5A. In certain embodiments, one gRNA can be complementary to a region harboring a mutation and another gRNA can be complementary to a region that does not harbor a mutation. For example, and not by way of limitation, one or more gRNAs can be complementary to a region harboring a mutation of the CTNNB1 gene and another gRNA can be complementary to a region not harboring a mutation of the CTNNB1 gene. For example, and not by way of limitation, one or more gRNAs can be complementary to a region harboring a mutation of the SLTM gene and another gRNA can be complementary to a region not harboring a mutation of the SLTM gene. In certain embodiments, one gRNA can be complementary to a region upstream of the genomic mutation and another gRNA can be complementary to a region down-stream of the genomic mutation.

In certain embodiments, one gRNA can be complementary to a region within one of the genes of the fusion gene and another gRNA can be complementary to a region within the other gene of the fusion gene. For example, and not by way of limitation, one gRNA can be complementary to a region within the SLC45A2 gene of the SLC45A2-AMACR fusion gene and another gRNA can be complementary to a region within the AMACR gene. In certain embodiments, one gRNA can be complementary to a region upstream of the chromosomal breakpoint of a fusion gene and another gRNA can be complementary to a region downstream of the chromosomal breakpoint. In certain embodiments, genome sequencing can be performed to determine the regions of the fusion gene that can be targeted by the gRNAs. In certain embodiment, the regions of the genes that are targeted by the gRNAs can be introns and/or exons.

In certain embodiments, the nucleic acid sequence encoding the Cas protein, e.g., Cas9, can be operably linked to a regulatory element, and when transcribed, the one or more gRNAs can direct the Cas protein to the target sequence in the genome and induce cleavage of the genomic loci by the Cas protein. In certain embodiments, the Cas9 protein cut about 3-4 nucleotides upstream of the PAM sequence present adjacent to the target sequence. In certain embodiments, the regulatory element operably linked to the nucleic acid sequence encoding the Cas protein can be a promoter, e.g., an inducible promoter such as a doxycycline inducible promoter. The term “operably linked,” when applied to DNA sequences, for example in an expression vector, indicates that the sequences are arranged so that they function cooperatively in order to achieve their intended purposes, i.e., a promoter sequence allows for initiation of transcription that proceeds through a linked coding sequence as far as the termination signal.

In certain embodiments, the Cas9 enzyme encoded by a vector of the present disclosure can comprise one or more mutations. The mutations may be artificially introduced mutations or gain- or loss-of-function mutations. Non-limiting examples of such mutations include mutations in a catalytic domain of the Cas9 protein, e.g., the RuvC and HNH catalytic domains, such as the D10 mutation within the RuvC catalytic domain and the H840 in the HNH catalytic domain. In certain embodiments, a mutation in one of the catalytic domains of the Cas9 protein results in the Cas9 protein functioning as a “nickase,” where the mutated Cas9 protein cuts only one strand of the target DNA, creating a single-strand break or “nick.” In certain embodiments, the use of a mutated Cas9 protein, e.g., Cas9D10A, allows the use of two gRNAs to promote cleavage of both strands of the target DNA. Additional non-limiting examples of Cas9 mutations include VP64, KRAB and SID4X, FLAG, EGFP and RFP. In certain embodiments, the genome editing technique of the present disclosure can further include introducing into the one or more cells an additional vector comprising a nucleic acid, that when expressed results in the death, e.g., apoptosis, of the one or more cells. In certain embodiments, this vector can further comprise one or more targeting sequences that are complementary (e.g., can hybridize) to the same and/or adjacent to the genomic sequences targeted by the gRNAs to allow homologous recombination to occur and insertion of the nucleic acid sequence (i.e., donor nucleic acid sequence) into the genome. In certain embodiments, the additional vector can further comprise one or more splice tag sequences of an exon/intron junction of a gene that makes up the fusion gene. In certain embodiments, the targeting sequences can be complementary to mutation site within a gene. In certain embodiments, one targeting sequence can be complementary to a region harboring a mutation within the gene targeted by the gRNAs and a second targeting sequence can be complementary to a region not harboring a mutation within the gene, to allow homologous recombination between the vector comprising the donor nucleic acid and the genome sequence cleaved by the Cas9 protein. In certain embodiments, the targeting sequences can be complementary to an intron, exon sequence and/or intron/exon splicing sequence within a gene of the fusion gene. In certain embodiments, one targeting sequence can be complementary to a region within one of the genes of the fusion gene targeted by the gRNAs and a second targeting sequence can be complementary to a region within the other gene of the fusion gene, to allow homologous recombination between the vector comprising the donor nucleic acid and the genome sequence cleaved by the Cas9 protein. For example, and not by way of limitation, one targeting sequence can be complementary to a region harboring a mutation of the CTNNB1 gene and another gRNA can be complementary to a region not harboring a mutation of the CTNNB1 gene. For example, and not by way of limitation, one targeting sequence can be complementary to a region harboring a mutation of the SLTM gene and another gRNA can be complementary to a region not harboring a mutation of the SLTM gene. In certain embodiments, one targeting sequence can be complementary to a region upstream of the genomic mutation and another targeting sequence can be complementary to a region down-stream of the genomic mutation. In certain embodiments, one targeting sequence can be complementary to a region upstream of the cleavage site generated by the Cas9 protein and another targeting sequence can be complementary to a region downstream of the mutation site. For example, and not by way of limitation, one targeting sequence can be complementary to a region within the SLC45A2 gene of the SLC45A2-AMACR fusion gene and another targeting sequence can be complementary to a region within the AMACR gene. In certain embodiments, one targeting sequence can be complementary to a region upstream of the cleavage site generated by the Cas9 protein and another targeting sequence can be complementary to a region downstream of the chromosomal breakpoint. Non-limiting examples of the types of nucleic acid sequences that can be inserted into the genome are disclosed above. In certain embodiments, the nucleic acid that is to be inserted into the genome encodes HSV-1 thymidine kinase. Additional non-limiting examples of nucleic acids and/or genes that can be inserted into the genome of a cell carrying a gene or fusion gene to induce cell death are set forth above.

The vectors for use in the present disclosure can be any vector known in the art. For example, and not by way of limitation, the vector can be derived from plasmids, cosmids, viral vectors and yeast artificial chromosomes. In certain embodiments, the vector can be a recombinant molecule that contains DNA sequences from several sources. In certain embodiments, the vector can include additional segments such as, but not limited to, promoters, transcription terminators, enhancers, internal ribosome entry sites, untranslated regions, polyadenylation signals, selectable markers, origins of replication and the like. In certain embodiments, the vectors can be introduced into the one or more cells by any technique known in the art such as by electroporation, transfection and transduction. In certain embodiments, the vectors can be introduced by adenovirus transduction.

### 5.7 Kits

The present disclosure further provides kits for treating a subject that carries one or more of the genomic mutations disclosed herein and/or for carrying out any one of the above-listed detection and therapeutic methods. In certain embodiments, the present disclosure provides kits for performing a targeted genome editing technique on one or more cancer cells within the subject that carries one or more genomic mutations disclosed herein.

Types of kits include, but are not limited to, packaged gene-specific probe and primer sets (e.g., TaqMan probe/primer sets), arrays/microarrays, antibodies, which further contain one or more probes, primers, or other reagents for detecting one or more genomic mutation and/or can comprise means for performing a genome editing technique.

In certain embodiments, the kit can include means for performing the genome editing techniques disclosed herein. For example, and not by way of limitation, a kit of the present disclosure can include a container comprising one or more vectors or plasmids comprising a nucleic acid encoding a Cas protein or a mutant thereof, e.g., Cas9^{D10A}. In certain embodiments, the nucleic acid encoding the Cas protein can be operably linked to a regulatory element such as a promoter. In certain embodiments, the one or more vectors can further comprise one or more gRNAs specific to a gene, e.g., specific to a gene mutation and/or sequences flanking the breakpoint of a gene mutation.

In certain embodiments, a kit of the present disclosure can include, optionally in the same container as the vector comprising the nucleic acid encoding a Cas protein or in another container, one or more vectors or plasmids comprising a nucleic acid, that when expressed (in the presence of absence of a compound) results in cell death. For example, and not by way of limitation, the nucleic acid sequence can encode the Herpes Simplex Virus 1 (HSV-1) thymidine kinase, Exotoxin A from Pseudomonas aeruginosa, Diphtheria toxin from Corynebacterium diphtheri, Ricin or abrin from Ricinus communi (castor oil plant), Cytosine deaminase from bacteria or yeast, Carboxyl esterase or Varicella Zoster virus (VZV) thymidine kinase. In certain embodiments, this vector can further comprise one or more targeting sequences that are complementary to sequences within the target gene to promote homologous recombination and insertion of the donor nucleic acid.

In certain embodiments, where the donor nucleic acid encodes HSV-1 thymidine kinase, the kit can further comprise ganciclovir and/or valganciclovir.

In certain non-limiting embodiments, a kit of the present disclosure can further comprise one or more nucleic acid primers or probes and/or antibody probes for use in carrying out any of the above-listed methods. Said probes may be detectably labeled, for example with a biotin, colorimetric, fluorescent or radioactive marker. A nucleic acid primer may be provided as part of a pair, for example for use in polymerase chain reaction. In certain non-limiting embodiments, a nucleic acid primer may be at least about 10 nucleotides or at least about 15 nucleotides or at least about 20 nucleotides in length and/or up to about 200 nucleotides or up to about 150 nucleotides or up to about 100 nucleotides or up to about 75 nucleotides or up to about 50 nucleotides in length. A nucleic acid probe may be an oligonucleotide probe and/or a probe suitable for FISH analysis. In specific non-limiting embodiments, the kit comprises primers and/or probes for analysis of at least one gene mutation. In certain embodiments, the kit comprises primers for analysis of CTNNB, SLTM, or SLC45A2-AMACR.

In certain non-limiting embodiments, the nucleic acid primers and/or probes may be immobilized on a solid surface, substrate or support, for example, on a nucleic acid microarray, wherein the position of each primer and/or probe bound to the solid surface or support is known and identifiable. The nucleic acid primers and/or probes can be affixed to a substrate, such as glass, plastic, paper, nylon or other type of membrane, filter, chip, bead, or any other suitable solid support. The nucleic acid primers and/or probes can be synthesized directly on the substrate or synthesized separate from the substrate and then affixed to the substrate. The arrays can be prepared using known methods.

In non-limiting embodiments, a kit provides nucleic acid probes for FISH analysis to determine the presence of one or more genomic mutation in a sample obtained from a subject.

In specific non-limiting embodiments, probes to detect a fusion gene may be provided such that separate probes each bind to the two components of the fusion gene, or a probe may bind to a “junction” that encompasses the boundary between the spliced genes. For example, and not by way of limitation, the junction is the region where the two genes are joined together. In specific non-limiting embodiments, the kit comprises said probes for analysis of at SLC45A2-AMACR.

### 5.8 Exemplary Embodiments

Embodiment 1. A genome editing method comprising
   (i) introducing a first polynucleotide encoding a suicide gene and a second polynucleotide encoding a nuclease into a cell, wherein the nuclease targets a genomic mutation of a gene selected from CTNNB1, SLTM, SLC45A2, AMACR, or a combination thereof, and
   (ii) contacting the cell with an agent capable of inducing killing of the cell.
Embodiment 2. The genome editing method of embodiment 1, wherein the suicide gene is selected from herpes simplex virus thymidine kinase (HSV-tk), Exotoxin A from Pseudomonas aeruginosa, Diphtheria toxin from Corynebacterium diphtheri, Ricin or abrin from Ricinus communi (castor oil plant), Cytosine deaminase, Carboxyl esterase, Varicella Zoster virus (VZV) thymidine kinase, or a combination thereof.
Embodiment 3. The genome editing method of embodiment 1 or 2, wherein the suicide gene is herpes simplex virus thymidine kinase (HSV-tk).
Embodiment 4. The genome editing method of embodiment 3, wherein the suicide gene comprises an amino acid sequence at least 80% identical to the amino acid sequence set forth in SEQ ID NO: 25.
Embodiment 5. The genome editing method of embodiment 3 or 4, wherein the suicide gene comprises the amino acid sequence set forth in SEQ ID NO: 25.
Embodiment 6. The genome editing method of any one of embodiments 1-5, wherein the first polynucleotide comprises a first homology recombination arm and a second homology recombination arm.
Embodiment 7. The genome editing method of embodiment 6, wherein the first homology recombination arm and the second homology recombination arm flank the first polynucleotide.
Embodiment 8. The genome editing method of any one of embodiments 1-7, wherein the nuclease is selected from a zinc finger nuclease, a meganuclease, a TALE nuclease, or a CRISPR system nuclease.
Embodiment 9. The genome editing method of any one of embodiments 1-8, wherein the nuclease is a CRISPR system nuclease.
Embodiment 10. The genome editing method of embodiment 8 or 9, wherein the nuclease is selected from Cas1, Cas1B, Cas2, Cas3, Cas4, Cas5, Cas6, Cas7, Cas8, Cas9 (also known as Csn1 or Csx12), Cas9^{D10A}, Cas1O, Csy1, Csy2, Csy3, Cse 1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, Cas9HiFi, or a combination thereof.
Embodiment 11. The genome editing method of any one of embodiments 8-10, wherein the nuclease is Cas9 or Cas9^{D10A} Embodiment 12. The genome editing method of any one of embodiments 8-11, further comprising one or more guide RNAs (gRNAs).
Embodiment 13. The genome editing method of embodiment 12, wherein the one or more gRNAs targets the genomic mutation.
Embodiment 14. The genome editing method of any one of embodiments 1-13, wherein the genomic mutation is a single nucleotide mutation or a single nucleotide polymorphism (SNP).
Embodiment 15. The genome editing method of any one of embodiments 1-14, wherein the genomic mutation is a mutation of an oncogene, a tumor suppressor gene, or an oncogenic fusion gene.
Embodiment 16. The genome editing method of any one of embodiments 1-15, wherein the genomic mutation is a mutation of the CTNNB1 gene.
Embodiment 17. The genome editing method of embodiment 16, wherein the mutation of the CTNNB1 gene is a S45P mutation of CTNNB1.
Embodiment 18. The genome editing method of any one of embodiments 1-15, wherein the genomic mutation is a mutation of the SLTM gene.
Embodiment 19. The genome editing method of embodiment 18, wherein the mutation of the SLTM gene is a V235G mutation of SLTM.
Embodiment 20. The genome editing method of any one of embodiments 1-15, wherein the genomic mutation is a breakpoint of a SLC45A2-AMACR fusion gene.
Embodiment 21. The genome editing method of any one of embodiments 1-15, wherein the genomic mutation is a mutation of the SLC45A2-AMACR fusion gene.
Embodiment 22. The genome editing method of any one of embodiments 1-21, wherein the first polynucleotide and the second polynucleotide are included in a vector.
Embodiment 23. The genome editing method of any one of embodiments 1-21, wherein the first polynucleotide is included in a vector and the second polynucleotide is included in a second vector.
Embodiment 24. The genome editing method of any one of embodiments 1-23, wherein the agent is selected from ganciclovir, valganciclovir, or a combination thereof.
Embodiment 25. The genome editing method of any one of embodiments 1-24, wherein the agent is ganciclovir.
Embodiment 26. A genome editing system comprising:
   (i) a first polynucleotide encoding a suicide gene;
   (ii) a second polynucleotide encoding a nuclease targeting a genomic mutation of a gene selected from CTNNB1, SLTM, SLC45A2, AMACR, or a combination thereof; and
   (iii) an agent capable of inducing killing of a cell expressing the suicide gene.
Embodiment 27. The genome editing system of embodiment 26, wherein the suicide gene is selected from herpes simplex virus thymidine kinase (HSV-tk), inducible Caspase 9 suicide gene (iCasp-9), truncated human epidermal growth factor receptor (EGFRt), or a combination thereof.
Embodiment 28. The genome editing system of embodiment 26 or 27, wherein the suicide gene is herpes simplex virus thymidine kinase (HSV-tk).
Embodiment 29. The genome editing system of embodiment 28, wherein the suicide gene comprises an amino acid sequence at least 80% identical to the amino acid sequence set forth in SEQ ID NO: 25.
Embodiment 30. The genome editing system of embodiment 28 or 29, wherein the suicide gene comprises the amino acid sequence set forth in SEQ ID NO: 25.
Embodiment 31. The genome editing system of any one of embodiments 26-30, wherein the first polynucleotide comprises a first homology recombination arm and a second homology recombination arm.
Embodiment 32. The genome editing system of embodiment 31, wherein the first homology recombination arm and the second homology recombination arm flank the first polynucleotide.
Embodiment 33. The genome editing system of any one of embodiments 26-32, wherein the nuclease is selected from a zinc finger nuclease, a meganuclease, a TALE nuclease, or a CRISPR system nuclease.
Embodiment 34. The genome editing system of any one of embodiments 26-33, wherein the nuclease is a CRISPR system nuclease.
Embodiment 35. The genome editing system of embodiment 33 or 34, wherein the nuclease is selected from Cas1, Cas1B, Cas2, Cas3, Cas4, Cas5, Cas6, Cas7, Cas8, Cas9 (also known as Csn1 or Csx12), Cas9^{D10A}, Cas1O, Csy1, Csy2, Csy3, Cse 1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, Cas9HiFi, or a combination thereof.
Embodiment 36. The genome editing system of any one of embodiments 33-35, wherein the nuclease is Cas9 or Cas9^{D10A} Embodiment 37. The genome editing system of any one of embodiments 33-36, further comprising one or more guide RNAs (gRNAs).
Embodiment 38. The genome editing system of embodiment 37, wherein the one or more gRNAs targets the genomic mutation.
Embodiment 39. The genome editing system of any one of embodiments 26-38, wherein the genomic mutation is a single nucleotide mutation or a single nucleotide polymorphism (SNP).
Embodiment 40. The genome editing system of any one of embodiments 26-39, wherein the genomic mutation is a mutation of an oncogene, a tumor suppressor gene, or an oncogenic fusion gene.
Embodiment 41. The genome editing system of any one of embodiments 26-40, wherein the genomic mutation is a mutation of the CTNNB1 gene.
Embodiment 42. The genome editing system of embodiment 41, wherein the mutation of the CTNNB1 gene is a S45P mutation of CTNNB1.
Embodiment 43. The genome editing system of any one of embodiments 26-40, wherein the genomic mutation is a mutation of the SLTM gene.
Embodiment 44. The genome editing system of embodiment 43, wherein the mutation of the SLTM gene is a V235G mutation of SLTM.
Embodiment 45. The genome editing system of any one of embodiments 26-40, wherein the genomic mutation is a breakpoint of a SLC45A2-AMACR fusion gene.
Embodiment 46. The genome editing system of any one of embodiments 26-40, wherein the genomic mutation is a mutation of the SLC45A2-AMACR fusion gene.
Embodiment 47. The genome editing system of any one of embodiments 26-46, wherein the first polynucleotide and the second polynucleotide are included in a vector.
Embodiment 48. The genome editing system of any one of embodiments 26-46, wherein the first polynucleotide is included in a vector and the second polynucleotide is included in a second vector.
Embodiment 49. The genome editing system of any one of embodiments 26-48, wherein the agent is selected from ganciclovir, valganciclovir, or a combination thereof.
Embodiment 50. The genome editing system of embodiment 49, wherein the agent is ganciclovir.
Embodiment 51. A composition comprising the genome editing system of any one of embodiments 26-50.
Embodiment 52. The composition of embodiment 51, wherein the composition is a pharmaceutical composition.
Embodiment 53. A kit comprising the genome editing system of any one of embodiments 26-50.
Embodiment 54. A kit comprising the composition of embodiment 51 or 52.
Embodiment 55. A method of treating a subject having a pre-malignant or neoplastic condition, the method comprising:
   (i) detecting, in a sample obtained from the subject, a genomic mutation of a gene selected from CTNNB1, SLTM, SLC45A2, AMACR, or a combination thereof; and
   (ii) administering an effective amount of a first polynucleotide encoding a suicide gene and a second polynucleotide encoding a nuclease targeting the genomic mutation.
Embodiment 56. The method of embodiment 55, further comprising administering an effective amount of an agent capable of inducing killing of a cell expressing the suicide gene.
Embodiment 57. The method of embodiment 55 or 56, wherein the pre-malignant or neoplastic condition is a condition of the liver.
Embodiment 58. The method of embodiment 57, wherein the condition of the liver is hepatocellular carcinoma (HCC).
Embodiment 59. A method of treating a subject having a cancer, the method comprising:
   (i) detecting, in a sample obtained from the subject, a genomic mutation of a gene selected from CTNNB1, SLTM, SLC45A2, AMACR, or a combination thereof; and
   (ii) administering an effective amount of a first polynucleotide encoding a suicide gene and a second polynucleotide encoding a nuclease targeting the genomic mutation.
Embodiment 60. A method of preventing, minimizing, and/or reducing the growth of a cancer in a subject, the method comprising:
   (i) detecting, in a sample obtained from the subject, a genomic mutation; and
   (ii) administering an effective amount of a first polynucleotide encoding a suicide gene and a second polynucleotide encoding a nuclease targeting a genomic mutation of a gene selected from CTNNB1, SLTM, SLC45A2, AMACR, or a combination thereof.
Embodiment 61. A method for lengthening the period of survival of a subject having cancer, the method comprising:
   (i) detecting, in a sample obtained from the subject, a genomic mutation of a gene selected from CTNNB1, SLTM, SLC45A2, AMACR, or a combination thereof; and
   (ii) administering an effective amount of a first polynucleotide encoding a suicide gene and a second polynucleotide encoding a nuclease targeting the genomic mutation.
Embodiment 62. A method for reducing the risk of, inhibiting, or preventing metastatic spread of a cancer in a subject, the method comprising:
   (i) detecting, in a sample obtained from the subject, a genomic mutation of a gene selected from CTNNB1, SLTM, SLC45A2, AMACR, or a combination thereof; and
   (ii) administering an effective amount of a first polynucleotide encoding a suicide gene and a second polynucleotide encoding a nuclease targeting the genomic mutation.
Embodiment 63. The method of any one of embodiments 59-62, further comprising administering an effective amount of an agent capable of inducing killing of a cell expressing the suicide gene.
Embodiment 64. The method of any one of embodiments 59-63, wherein the cancer is a liver cancer.
Embodiment 65. The method of embodiment 64, wherein the liver cancer is hepatocellular carcinoma (HCC).
Embodiment 66. The method of any one of embodiments 55-65, wherein the genomic mutation is a single nucleotide mutation or a single nucleotide polymorphism (SNP).
Embodiment 67. The method of any one of embodiments 55-66, wherein the genomic mutation is a mutation of an oncogene, a tumor suppressor gene, or an oncogenic fusion gene.
Embodiment 68. The method of any one of embodiments 55-67, wherein the genomic mutation is a mutation of the CTNNB1 gene.
Embodiment 69. The method of embodiment 68, wherein the mutation of the CTNNB1 gene is a S45P mutation of CTNNB1.
Embodiment 70. The method of any one of embodiments 55-67, wherein the genomic mutation is a mutation of the SLTM gene.
Embodiment 71. The method of embodiment 70, wherein the mutation of the SLTM gene is a V235G mutation of SLTM.
Embodiment 72. The method of any one of embodiments 55-67, wherein the genomic mutation is a breakpoint of a SLC45A2-AMACR fusion gene.
Embodiment 73. The method of any one of embodiments 55-67, wherein the genomic mutation is a mutation of the SLC45A2-AMACR fusion gene.
Embodiment 74. The method of any one of embodiments 56-58 or 63-73, wherein the agent is selected from ganciclovir, valganciclovir, or a combination thereof.
Embodiment 75. The composition of embodiment 51 or 52 or the kit of embodiment 53 or 54 for use in treating a subject having a pre-malignant or neoplastic condition, treating a subject having a cancer, preventing, minimizing, and/or reducing the growth of a cancer in a subject, lengthening the period of survival of a subject having cancer, or reducing the risk of, inhibiting, or preventing metastatic spread of a cancer in a subject.
Embodiment 76. Use of the composition of embodiment 51 or 52 or the kit of embodiment 53 or 54 for the manufacturing of a medicament for treating a subject having a pre-malignant or neoplastic condition, treating a subject having a cancer, preventing, minimizing, and/or reducing the growth of a cancer in a subject, lengthening the period of survival of a subject having cancer, or reducing the risk of, inhibiting, or preventing metastatic spread of a cancer in a subject.

### 6. Examples

The presently disclosed subject matter will be better understood by reference to the following Example, which are provided as exemplary of the presently disclosed subject matter, and not by way of limitation.

### 6.1 Results

Targeting the CTNNB1^{S45P} mutation in vitro and in vivo

CTNNB1 mutations are frequent in the genome of HCC and can range from 26-37% of all HCC cases¹⁵. Among the mutation profile of CTNNB1, the mutation that converts serine at position 45 to proline is one of the frequent mutations in human HCC samples. Such mutation has been shown to drive HCC development in mice¹⁶. To investigate whether S45P mutation is targetable by Cas9-mediated genome editing, a pair of gRNAs were designed specifically for thymidine to cytosine mutation at the position of 133 of the coding sequence of CTNNB1, which resulted in the conversion of serine to proline. As shown in FIG. 1A, the application of gRNA- and spCas9 cleaved a 409 bp mutant (C. 133T>C) CTNNB1 cDNA into 265 and 144 bp fragments, respectively, while the same application had no impact on the wild-type cDNA. On the other hand, the application of gRNA+ and spCas9 resulted in the cleavage of both mutant and wild-type CTNNB1 templates into fragments of 226 and 183 bp. These results indicate that gRNA-is highly specific for CTNNB1 mutant. Next, a recombinant adenovirus was constructed containing said gRNAs and a donor unit that contained sequences mCherry-HSV1-tk sandwiched by cDNA sequences upstream and downstream of the targeted sequence (FIG. 1B). The cassette of mCherry-HSV1-tk contained no promoter but retained the ribosome binding site and an intact ATG translation start site. Thus, mCherry-HSV1-tk was not expressed unless the cassette was inserted into an active transcribed position in the genome. The recombinant adenovirus was then co-infected with a recombinant adenovirus expressing Cas9^{D10A}-EGFP into HUH7 cells that were transformed with a pT3-EF 1a-CTNNB1^{S45P} expression vector. As shown in FIGS. 1B and 1C, the co-introduction of ad-Cas9^{D10A}-EGFP and ad-CTNNB1-mCherry-tk-gRNA induced strong expression of mCherry-HSV1-tk in HUH7 cells expressing CTNNB1^{S45P}with expression frequency reaching 77.8% of the infected cells. On the other hand, the expression of mCherry-HSV1-tk was minimal when HUH7 cells were transformed with the wild-type CTNNB1 cDNA construct. In the absence of gRNA, little expression of mCherry-HSV1-tk was detected in CTNNB1^{S45P}-transformed HUH7 cells. These results indicated that insertion of mCherry-HSV1-tk into the cancer genome by these reagents was CTNNB1^{S45P} mutation dependent.

To investigate whether these genome targeting reagents have therapeutic effect, pT3-CTNNB1^{S45P} was hydrodynamically injected into the tail vein of FVB/NJ mice along with pT3-HMET and pSB (plasmid sleeping beauty) to induce spontaneous liver cancer. As shown in FIG. 2A, numerous small liver cancer nodules began to appear in magnetic resonance imaging (MRI) two months after the injection. In the ninth week, a cocktail of genome targeting reagents, including pCas9^{D10A}-EGFP and pCTNNB1-mCherry-tk-gRNA, mixed with in vivo-JetPEI delivery reagents were injected into the tail vein of the animals three times a week for HSV1-tk insertion into the cancer genome. This was coupled with an intraperitoneal injection of pro-drug ganciclovir for cancer treatment. The progression of the liver cancers was monitored through MRI imaging in a weekly fashion. As shown in FIGS. 2A and 2B, there was a gradual reduction of tumor volume after 4 weeks of treatment. In contrast, the control-treated (pCas9^{D10A}-EGFP/pCTNNB1-mCherry-tk, no gRNA) group experienced a 3.9 fold increase in tumor volume in the same period. All control-treated animals died in 90 days after the induction of liver cancer, while all the animals treated with the therapeutic reagents survived beyond this period (FIG. 2C, p=0.0004).

To investigate whether CTNNB1^{S45P} targeting reagents also worked similarly in human cancers that contained CTNNB1^{S45P} mutation, HUH7 cells were transfected with pT3-EF1a-CTNNB1^{S45P}-FLAG. HUH7 cells positive for CTNNB1^{S45P}-FLAG expression were xenografted into the subcutaneous region of severe combined immunodeficiency mice. When the xenografted cancers reached an average of 229 mm³, the CTNNB1^{S45P} targeting therapeutic reagents were applied through the tail vein injection as described above. As shown in FIG. 2D, the treatment of the CTNNB1^{S45P} targeting limited the growth of HUH7 cells harboring the mutation, while the control-treated groups experienced exponential growth of cancer. On the other hand, HUH7 cells harboring the wild-type CTNNB1 did not respond to the CTNNB1^{S45P} targeting therapeutic reagents, as the tumor volumes of the treated groups did not show appreciable reduction versus those of the controls. All seven animals xenografted with HUH7 cells containing CTNNB1S45P and treated with including pCas9^{D10A}EGFP and pCTNNB1-mCherry-tk-gRNA had no incidence of metastasis (FIG. 2E). On the other hand, the CTNNB1^{S45P} HUH7 xenografted animals treated with control reagents had a 62.5% (⅝, p=0.03) rate of metastasis. Animals xenografted with HUH7 containing WT CTNNB1 had similar rates of metastases ( 4/8 vs ⅜, p=1). All animals xenografted with HUH7 harboring CTNNB1^{S45P} and treated with the control therapeutic reagents died less than 4 weeks after the tumor xenografting, while 6/7 animals treated with CTNNB1^{S45P}targeting reagents survived through 42 days. In contrast, the mortality rate of animals xenografted with HUH7 cells harboring the WT CTNNB1 showed minimal difference in mortality (⅝ versus 6/8 mortality) between the treated and the control group. These results indicated that mutation targeting at CTNNB1^{S45P} was highly specific and was mutation dependent.

### Targeting the Breakpoint of SLC45A2-AMACR

SLC45A2-AMACR gene fusion occurs frequently in human cancers. The rate of occurrence of this fusion gene in HCC reaches 78.6%¹⁷. Interestingly, all HCC cell lines positive for SLC45A2-AMACR had identical breakpoints for the fusion in their genomes. To investigate whether the breakpoint of SLC45A2-AMACR is targetable by Cas9-mediated insertion of HSV1-tk, a pair of gRNA was designed to direct the cutting in the breakpoint region of the cancer genome by Cas9. As shown in FIG. 3A, the application of gRNA- to the digestion mix of spCas9 cut the breakpoint containing DNA fragment of 4.1 kb into 2.6 and 1.5 kb, while the gRNA+cleaved the same fragment into 2.5 and 1.6 kb, matching the expected cutting position generated by these gRNAs. To investigate these gRNA-induced insertions of HSV1-tk into the breakpoint region of SLC45A2-AMACR in vivo, a donor-gRNA construct was generated by ligating a 956 bp DNA corresponding to exon 2 and intron 2 of SLC45A2 with tk-mCherry, followed by 877 bp of intron 1 and exon 2 from AMACR (FIG. 3B). The construct was packaged into ad5 to create a recombinant adenovirus (ad-SLAM-tk-mCherry-gRNA) containing the donor cassette and expressing the gRNAs. Ad-SLAM-tk-mCherry-gRNA was then applied to co-infect with a recombinant adenovirus that expressed Cas9^{D10A}-EGFP into HEPG2, HUH7, and DU145 cells. As shown in FIGS. 3B and 3C, the co-infection induced strong expression of HSV1-tk-mCherry in SLC45A2-AMACR positive HEPG2 and HUH7 cells, with 98.7 and 95.10% of HEPG2 and HUH7 cells being positive for HSV1-tk-mCherry, respectively. In contrast, DU145, a cell line negative for SLC45A2-AMACR fusion, had only 3.3% positive for HSV1-tk-mCherry expression. These results indicated that the insertion of HSV1-tk-mCherry into the genome is specific to the breakpoint of SLC45A2-AMACR gene fusion.

To investigate whether targeting SLC45A2-AMACR is feasible in spontaneous liver cancer model, a SLC45A2-AMACR cDNA with a full breakpoint intron to mimic the genome structure of SLC45A2-AMACR gene fusion was constructed. This intron-containing cDNA was constructed into a pT3-Ela vector to create pT3-SLC45A2^{exon-2-2}-breakpoint intron-AMACR^{exon2-6}-FLAG. The construct was then hydrodynamically injected with pSB into the tail vein of ptenflox mice where Pten was somatically disrupted in the hepatocytes through intraperitoneal application of AAV8-cre. Spontaneous liver cancer was detected in 12 weeks after the introduction of pT3-SLC45A2-breakpoint intron-AMACR-FLAG (FIG. 4A). The treatment with SLC45A2-AMACR breakpoint targeting and Cas9^{D10A}-EGFP expression constructs was applied using in vivo JetPEI delivery system the following week. As shown in FIGS. 4A and 4B, a gradual reduction of tumor burden was found for cancers treated with the constructs of pCas9^{D10A}-EGFP and pSLAM-HSV1-tk-mCherry-gRNA. On the other hand, when the liver cancers were treated with pCas9^{D10A}-EGFP or pSLAM-HSV1-tk-mCherry-gRNA alone, the tumor progressed significantly, reaching 18.2 (p<0.01) and 12.1 (p<0.01) fold of the treatment group, respectively. All animals died from cancer in 4 weeks if not treated with the correct targeting reagents, while all the animals survived the same period when treated with the correct targeting reagents (p=0.0016, FIG. 4C).

To investigate whether the SLC45A2-AMACR genome targeting reagents were also effective in human cancer cell lines that contained the gene fusion, HEPG2, which was positive for SLC45A2-AMACR, was xenografted to SCID mice subcutaneously. When the average tumor size of HEPG2 reached 167 mm³, a cocktail of in vivo JetPEI containing the constructs of Cas9^{D10A}-EGFP and pSLAM-HSV1-tk-mCherry-gRNA was injected into the tail-vein of the mice three times a week. As shown in FIG. 4D, the treatment reduced the tumor burden by an average of 9.5 fold (p<0.01) in comparison with the pSLAM-HSV1-tk-mCherry-gRNA only controls, or of 7.3 fold (p<0.01) with Cas9^{D10A}-EGFP only controls. No incidence of metastasis or invasion was found for the animals treated with Cas9^{D10A}-EGFP and pSLAM-HSV1-tk-mCherry-gRNA. In contrast, the incidence of metastasis/invasion reached 100% for Cas9^{D10A}-EGFP only (p<0.01) and 80% for pSLAM-HSV1-tk-mCherry-gRNA only control group (p<0.05, FIG. 4E). All mice in the control groups died less than 42 days after the xenografting, while all mice in the treatment group survived the same period (FIG. 4F). Similar effects were seen with the constructs of pCas9^{D10A}-EGFP and pSLAM-HSV1-tk-mCherry-gRNA on HUH7 xenografted SCID mice. The treatment reduced the tumor burden by an average of 17.6 fold (p<0.01, FIG. 4G) and decreased the incidence of metastasis/invasion by 4 fold (p<0.05, FIG. 4H). The control-treated mice xenografted with HUH7 died less than 35 days after the xenografting, while all the mice in the treatment group survived through 42 days (p=0.0018, FIG. 4I). These results suggest that SLC45A2-AMACR genome-targeted treatment may be an effective approach to treating liver cancers.

### Targeting Naturally Occurring Mutations of Unknown Significance

The flexibility of Cas9-mediated genome targeting makes it feasible to target a wide variety of mutations. Many mutations in cancer cells may not be cancer drivers but nonetheless play important roles in assisting cancer development. Inclusion of these mutations in targeting not only increases the repertoire of targets against cancer cells but also provides a new approach to counter genome heterogeneity of liver cancer. To investigate the flexibility of mutation targeting, the exome and transcriptome of HUH7 cells were sequenced. Six hundred and fourteen single nucleotide polymorphisms (SNP) in HUH7 genome were matched in both mRNA and exome levels and were identified as the pathological mutations defined in the COSMIC database. One of the SNPs occurred in a gene called SAFB-like transcription modulator (SLTM). The SNP was located in exon 7. The SNP converted valine at 235 of SLTM to glycine (C. 704 T>G, Hg19-Chr15: 58899823). Interestingly, two additional SNPs were found in a nearby region (C. 697A>G, HG19-Chr15: 58899830, and C. 694C>A, Hg19-Chr15: 58899833) of SLTM. The mutation of C. 704 T>G produced a potential new PAM sequence for Cas9. A gRNA was designed encompassing mutations C. 694C>A and C. 697A>G, and utilized the mutation C. 704 T>G as a part of the PAM sequence. As shown in FIG. 5A, the gRNA specific for the mutations resulted in cleavage of mutant DNA but had no impact on the wild-type DNA. Next, a DNA fragment was constructed to express these gRNAs and contain a promoterless mCherry-HSV1-tk sandwiched by 954 bp from intron 6 and the 5′ end of exon 7 of SLTM and 908 bp from the 3′ end of exon 7 and intron 7 of SLTM (FIG. 5B). This construct was transfected into HUH7 cells that were infected with ad-Cas9^{D10A}-EGFP. As shown in FIGS. 5B and 5C, the transfection resulted in the co-expression of Cas9^{D10A}-EGFP and HSV1-tk-mCherry, indicating the insertion of HSV1-tk-mCherry into the target site. The insertion/expression rate of HUH7 cells reached 51.4% (FIG. 5C). In contrast, the similar treatment of SNU449 cells, which were negative for these mutations, resulted in the expression of Cas9^{D10A}-EGFP in most cells, but only 3.9% of cells expressed mCherry-HSV1-tk. These results indicated that the expression of HSV1-tk-mCherry was dependent on these specific mutations of SLTM. To investigate whether these mutation-targeting reagents had therapeutic impact, SCID mice were xenografted with HUH7 cells. These mice were then treated with a cocktail of in vivo JetPEI containing pCas9^{D10A}-EGFP and pSLTM-mCherry-tk-gRNA three times a week after the tumors reached an average size of 114 mm³. As shown in FIG. 5D, the treatment of these reagents resulted in smaller tumor volumes by 6 fold in comparison with pSLTM-mCherry-tk-gRNA control (p<0.01), and 5.3 fold with Cas9^{D10A}-EGFP only control (p<0.01). There was no incidence of metastasis/invasion in the treatment group. In contrast, all mice treated with the control reagents had events of metastasis/invasion (p<0.01, FIG. 5E). All control-treated animals succumbed to the xenografted HUH7 tumors in 32 days after the xenografting, while all the mice treated with the correct combination of reagents survived beyond this period (p=0.0018, FIG. 5F). Taken together, these results indicate that genome targeting is effective in treating cancers that contain targetable mutations.

### 6.2 Discussion

Genome editing technology has been known for decades. However, the utility of genome editing technology was limited until precision editing was discovered in the CRISPR-Cas9 system. Previous studies had shown that chromosome rearrangement and point mutation in cancer were targetable through Cas9 editing^{14,18}. However, no study had been performed on single nucleotide mutation targeting in liver cancer. The present disclosure showed for the first time that the insertion of a suicide gene into a point mutation through Cas9 editing achieved effective cancer treatment in both spontaneous and xenografted liver cancer models. One of the mutations occurred in CTNNB1, one of the most frequently mutated proto-oncogenes in liver cancer, while the other mutation occurred in SLTM, a regulator of RNA processing. Targeting at either mutation have achieved a high frequency of suicide gene insertion into the cancer genome and achieved partial remission of liver cancers that harbored these mutations. The present disclosure serves as a proof of principle in mutation targeting for human liver cancer.

The options for treating HCC of advanced stages remain few. Many of the small molecule treatments are minimally discriminatory towards normal and cancer cells. Thus, they may produce significant side effects. The Cas9-mediated insertion of a suicide gene such as HSV1-tk described in the present disclosure is mutation or chromosome rearrangement specific. In all three mutation/genome rearrangement targeting, the non-specific expression of HSV1-tk in non-targeted cells was less than 5 percent. As a result, these treatments may have minimal impact on the healthy tissues. In general, mutations and chromosome rearrangement underlie the development of human cancers. The number of mutations and chromosome rearrangements may increase along the progression of liver cancer. Thus, the number of targetable mutations and chromosome rearrangements may increase. Flexibility in targeting may be required in controlling the progression of cancer. The versatility of Cas9-mediated mutation targeting may be well-suited for such a task.

The efficiency of suicide gene insertion into the mutation sites appeared variable, ranging from 54% to 98%, depending on the target site and cancer cells. The mechanism for such variability remains unclear. It is possible that the loci of the genome with active transcription may be more accessible to Cas9 nuclease and gRNA molecules due to the unwinding structure of the genome loci. In addition, the tertiary structure of the chromosome may impact the chromosome recombination that is required for the insertion of the suicide gene. Despite the high efficiency of insertion, the treatment did not completely eliminate the cancer in either spontaneous or xenografted cancer model, suggesting residual cancer may evolve to develop a mechanism to resist the single target treatment. Further analysis is needed to understand the mechanism that evades the Cas9-mediated genome insertion of the suicide gene.

### 6.3 Conclusion

The present disclosure demonstrates a technology for mutation site insertion of a suicide gene (HSV1-tk) based on Cas9-mediated genome editing to treat liver cancers. The present disclosure shows the genome editing strategy applied to three different mutations: S45P mutation of CTNNB1, chromosome breakpoint of SLC45A2-AMACR gene fusion, and V235G mutation of SLTM. The results showed that the HSV1-tk insertion rate at the S45P mutation site of CTNNB1 reached 77.8%, while the insertion rates at the breakpoint of SLC45A2-AMACR gene fusion were 95.1-98.7%, and the insertion at V235G of SLTM 51.4%. When these targeting reagents were applied to treat mouse spontaneous liver cancer induced by CTNNB1S45P or SLC45A2-AMACR, the mice experienced reduced tumor burden and increased survival rate. Similar results were also obtained for the xenografted liver cancer model: significant reduction of tumor volume, reduction of metastasis rate, and improved survival were found in mice treated with the targeting reagent, in comparison with the control-treated groups. These results indicate that mutation targeting may hold promise as a versatile and effective approach to treating liver cancers.

### 6.4 Materials and Methods

### Cell Lines

The cell lines used in the study were purchased from the American Type Culture Collection (ATCC, Manassas, Virginia) and were cultured and maintained following the recommendations of the manufacturer. Cells were authenticated every 6 months and were free of mycoplasma.

### Construction of Vectors

To construct CTNNB1 expression vector, cDNA of CTNNB1 was obtained by PCR using AccuPrime™ Pfx DNA Polymerase (Invitrogen) with a pair of primers (gtcgacCACCATGGAGCAAAAGCTCATTTCTGAAGAGGACTTG [SEQ ID NO: 1)]/gcggccgcTTACAGGTCAGTATCAAAC [SEQ ID NO: 2]) corresponding to the CDS region of CTNNB1 with c-myc tag from Origene Inc. in the following condition: 94o C for 1 min, then 35 heating cycles at 95° C. for 15 seconds, 65° C. for 30 seconds, and 72° C. for 10 minutes. The PCR product was then restricted with Sa11 and Not1, and ligated into the similarly digested pT3-EF1a vector. The 5′ untranslated sequence of CTNNB1 was obtained by PCR with a pair of primers (tttaaaAGGATACAGCGGCTTCTGCGCG [SEQ ID NO: 3/gtcgacCACGCTGGATTTTCAAAACAG [SEQ ID NO: 4]) on cDNA obtained from a liver organ donor in the same conditions as mentioned above. The PCR product was digested with Dra1 and Sa11, and ligated to the similarly restricted vector created from the first step to create pT3-EF1a-CTNNB1 vector. To create a S45P mutation, 2-step mutational PCRs were performed: a PCR was performed with mutagenic primers (tttaaaAGGATACAGCGGCTTCTGCGCG [SEQ ID NO: 3]/GCCTTTACCACTCAGAGgAGGAGCTGTGGTAGT [SEQ ID NO: 5]) using AccuPrime™ Pfx DNA Polymerase in the following condition: 94° C. for 1 min, then 35 heating cycles at 95° C. for 15 seconds, 65° C. for 30 seconds, and 72° C. for 10 minutes. A separate PCR was performed using primers CACTACCACAGCTCCTcCTCTGAGTGGTAAAGGCAATC (SEQ ID NO: 6)/gcggccgcTTACAGGTCAGTATCAAAC (SEQ ID NO: 2). A final PCR was then performed on the mixture of 2 PCR products using primers tttaaaAGGATACAGCGGCTTCTGCGCG (SEQ ID NO: 3) and gcggccgcTTACAGGTCAGTATCAAAC (SEQ ID NO: 2) to obtain the mutant cDNA. The mutant product was digested with Dra1 and Not1, and ligated into the similarly digested pT3-EF1a vector to create pT3-EF1a-CTNNB1^{S45P} construct.

To construct CTNNB1-HSV1tk-RA-gRNA−/gRNA+vector, a chimera mCherry-2A-HSV1-tk coding sequence was synthesized. An 815 bp 5′ cDNA from CTNNB1 was then ligated to the upstream of the synthetic chimera DNA. Subsequently, a 718 bp 3′ cDNA from CTNNB1 was ligated downstream to the synthetic chimera DNA. The donor vector was completed by insertion of U6-gRNA-U6-gRNA+synthetic DNA block downstream to the 3′ cDNA of CTNNB1.

To construct pT3-EF1a-SLC45A1^{exon-2-2}-breakpoint intron-AMACRexon²⁻⁶, the 4031 bp DNA that included exons 1-2 of SLC45A2, followed by 1117 bp breakpoint intron and exons 2-6 of AMACR was chemically synthesized (BioBasic, Inc). The construct was then ligated into pT3-EF1a. To construct pSLAM-HSV1-tk-mCherry-gRNA vector, a fragment of 4167 bp DNA containing 956 bp from exon 2 and intron 2 of SLC45A2, a chimera coding sequence of HSV1-tk-2A-mCherry, 877 bp DNA from intron 2 and exon 3 of AMACR, was chemically synthesized. A unit of U6-gRNA-U6-gRNA+was then inserted downstream to the synthetic DNA to complete the construction. To construct pSLTM-mCherry-tk-gRNA vector, a 4796 bp donor DNA fragment was synthesized to include a 954-bp fragment of exon7 and intron 7 of SLTM, a chimera coding sequence for mCherry-2A-HSV1-tk, a 908-bp fragment of intron 7 and exon 8 of SLTM, followed by a unit of U6-gRNA-U6-gRNA+, was chemically synthesized. Cas9^{D10A}-EGFP construct was obtained from Addgene, inc.

### In Vitro Cas9 Target Cleavage Assays

gRNA DNA sequence plus scaffold DNA sequence for + or − DNA strand were amplified from the all-in-one vector with the following primers: GGCCAGTGAATTGTAATACGACTCACTATAGGGAGGCGGAAAGGCAATCCTGAG GAAG (SEQ ID NO: 7)/AAAAAAAGCACCGACTCGGTGCCACTTTTTC (SEQ ID NO: 8) for gRNA+template of CTNNB1, GGCCAGTGAATTGTAATACGACTCACTATAGGGAGGCGGTCTACCACAGCTCCTC CTCTG (SEQ ID NO: 9)/AAAAAAAGCACCGACTCGGTGCCACTTTTTC (SEQ ID NO: 8) for gRNA-template of CTNNB1, GGCCAGTGAATTGTAATACGACTCACTATAGGGAGGCGGGGTGCTAAACTTTTTC GTGA (SEQ ID NO: 10)/AAAAAAAGCACCGACTCGGTGCCACTTTTTC (SEQ ID NO: 8) for gRNA+template of SLC45A2-AMACR, GGCCAGTGAATTGTAATACGACTCACTATAGGGAGGCGGGAGAGCTCCCATTTTC CTCC (SEQ ID NO: 11)/AAAAAAAGCACCGACTCGGTGCCACTTTTTC (SEQ ID NO: 8) for gRNA-template of SLC45A2-AMACR, GGCCAGTGAATTGTAATACGACTCACTATAGGGAGGCGGCTGTGTGATCAGCCTC AGCT (SEQ ID NO: 12)/AAAAAAAGCACCGACTCGGTGCCACTTTTTC (SEQ ID NO: 8) for gRNA+template of SLTM, and GGCCAGTGAATTGTAATACGACTCACTATAGGGAGGCGGAGATGGAAGCTAATG CGACT (SEQ ID NO: 13)/AAAAAAAGCACCGACTCGGTGCCACTTTTTC (SEQ ID NO: 8) for gRNA-template of SLTM.

The PCR products were in vitro transcribed using In Vitro Transcription kit from Ambion, CA, to obtain gRNA+ and gRNA-products. Cleavage assays were performed at 25o C for 10 min and then 37° C. for 1 hour under the following condition: lx Cas9 nuclease reaction buffer, 30 nM gRNA 3 nM DNA template, and 30 nM Cas9 Nuclease, S. pyogenes. The cleaved DNA was visualized in 1% agarose gel electrophoresis.

### Fluorescence-Activated Cell Sorting (FACS) Analysis of Apoptotic Cells

The assays were previously described¹⁹⁻²⁷. Briefly, the cells treated with Ad-Cas9^{D10A}-EGFP/Ad-mCherry-tk Ad-CTNNB1-mCherry-tk-gRNA, or Ad-Cas9^{D10A}-EGFP/ad-SLAM-HSV1-tk-mCherry-gRNA or pCas9^{D10A}-EGFP/pSLTM-mCherry-HSV1-tk were trypsinized and washed twice with cold PBS. The cells were analyzed at the fluorescence emission at 610 nm (mCherry) and 509 nm (EGFP), respectively. The negative control, cells without treated reagents in the incubation medium, was used to set the background for the acquisition. WinMDI 2.9 software (freeware from Joseph Trotter) was used to analyze the data.

Mice and Hydrodynamic tail vein injections Hydrodynamic tail vein injections were performed as described previously^{28,29}. Briefly, first, PtentmlHwu/J mice of which exon 5 of Pten gene was flanked by loxP sites was treated with adeno-associated virus-cre (1×10¹⁰ PFU) through intra-peritoneal injection to create Pten knockout in most hepatocytes. Next, 20 μg of pT3-SLC45A2^{exon1-2}-breakpoint intron-AMACR^{exOn2-6}-FLAG, along with the sleeping beauty transposase (SB) in a ratio of 25:1 were diluted in 2 ml of normal saline (0.9% NaCl), filtered through 0.22 μm filter (Millipore), and injected into the lateral tail vein in 5 to 7 seconds. Mice were housed, fed, and monitored in accordance with the protocols approved by the Institutional Animal Care and Use Committee at the University of Pittsburgh School of Medicine. A similar hydrodynamic tail vein injection was performed with pT3-EF1a-CTNNB1^{S45P}/HMET injection except 20 pg for pHMET and pT3-EF1a-CTNNB1^{S45P} each on FVB/NJ mice.

### Tumor Growth and Spontaneous Metastasis

The sample size of tumor xenografting analysis was based on power analysis, assuming 90% survival for treated animals and 10% for control treated. Complete blindness was applied in the animal study. The xenografting procedure was described previously^{21,25-27,30-32}. Briefly, approximately 5×10⁶ viable HUH7-CTNNB1, HUH7-CTNNB1^{S45P}, HUH7 or HEPG2 cells, suspended in 0.2 mL of Hanks' balanced salt solution (Krackeler Scientific, Inc., Albany, NY) were subcutaneously implanted in the abdominal flanks of SCID mice to generate one tumor per mouse. The breakdown of the treated groups is the following: 6 for HUH7 cells transformed with pT3-EF1a-CTNNB1^{S45P} and treated with pCTNNB1-mCherry-tk-gRNA/pCas9^{D10A}-EGFP and ganciclovir; 6 for HUH7 cells transformed with pT3-EF1a-CTNNB1 and treated with pCTNNB1-mCherry-tk-gRNA/pCas9^{D10A-EGFP and ganciclovir (control);} 6 for HUH7 cells transformed with pT3-EF1a-CTNNB1S45P and treated p CTNNB1-mCherry-tk (no gRNA)/pCas9^{D10A}-EGFP and ganciclovir (control); 6 for HUH7 cells transformed with pT3-EF1a-CTNNB1 and treated with pCTNNB1-mCherry-tk (no gRNA)/pCas9^{D10A}-EGFP and ganciclovir (control). For SLC45A2-AMACR targeting, the distribution is the following: 10 mice for HEPG2 cells and treated with pSLAM-tk-mCherry-gRNA/pCas9^{D10A}-EGFP and ganciclovir, 5 for HEPG2 cells and treated with pCas9^{D10A-EGFP and ganciclovir,} 5 for HEPG2 cells and treated with pSLAM-tk-mCherry-gRNA and ganciclovir, 5 for HUH7 cells and treated with pSLAM-tk-mCherry-gRNA/pCas9^{D10A-EGFP and ganciclovir,} 5 for HUH7 and treated with pSLAM-tk-mCherry (no gRNA)/pCas9^{D10A}-EGFP and ganciclovir. For SLTM mutation targeting, the distribution is the following: 5 mice for HUH7 and treated with pSLTM-mCherry-tk-gRNA/pCas9^{D10A}-EGFP and ganciclovir, 5 mice for HUH7 and treated with pSLTM-mCherry-tk-gRNA and ganciclovir, 5 mice for HUH7 and treated with pSLTM-mCherry-tk-gRNA and ganciclovir. Mice were observed daily, and their body weight and tumor size were recorded weekly. Three weeks after xenografting, these mice were treated with the indicated cocktail of in vivo JetPEI with the proportion based on the recommendation from the manufacturer (Genesee Scientific, Inc) and ganciclovir (80 mg/kg) 3 times a week through tail vein injection. After 42 days, mice in the treatment groups were killed, and necropsies were performed. For mice treated with control reagents, necropsies were performed when mice died from the xenografted cancers. The protocol of animal experiments is approved by Institutional Review Board of University of Pittsburgh.

### Magnetic Resonance Imaging

Mice were anesthetized via a nose cone with 1-2% isoflurane and 02, they were then positioned on an animal bed with the abdomen secured to reduce motion artifacts, and placed in the scanner. Respiration rate was monitored, and body temperature was maintained using a warm air heating system, (SA Instruments, New York, NY, USA). MRI was performed on a 7T/30-cm AVIII spectrometer (Bruker Biospin, Billerica, MA) equipped with a 12 cm gradient set and using a 40 mm quadrature RF volume coil, and Paravision 6.0.1. A T2-weighted RARE sequence was used to visualize the liver tumors, with the following parameters: repetition time (TR)/echo time (TE)=5000/24 ms, field of view (FOV) of 35 mm², acquisition matrix=256×256, 30-47 slices (depending on liver tumor size) with a slice thickness of 0.7 mm, 8 averages, and a RARE factor=8.

### 6.5 References

1. Siegel, R. L., Miller, K. D., Wagle, N. S. & Jemal, A. Cancer statistics, 2023. CA: a cancer journalfor clinicians 73, 17-48, doi:10.3322/caac.21763 (2023).
2. Sung, H. et al. Global cancer statistics 2020: GLOBOCAN estimates of incidence and mortality worldwide for 36 cancers in 185 countries. CA: a cancer journal for clinicians, doi:10.3322/caac.21660 (2021).
3. Mehta, N. Hepatocellular Carcinoma-How to Determine Therapeutic Options. Hepatol Commun 4, 342-354, doi:10.1002/hep4.1481 (2020).
4. Brar, G. et al. Hepatocellular Carcinoma Survival by Etiology: A SEER-Medicare Database Analysis. Hepatol Commun 4, 1541-1551, doi:10.1002/hep4.1564 (2020).
5. Khemlina, G., Ikeda, S. & Kurzrock, R. The biology of Hepatocellular carcinoma: implications for genomic and immune therapies. Mol Cancer 16, 149, doi:10.1 186/s12943-017-0712-x (2017).
6. Luo, J. H. et al. Transcriptomic and genomic analysis of human hepatocellular carcinomas and hepatoblastomas. Hepatology (Baltimore, Md 44, 1012-1024 (2006).
7. Nalesnik, M. A. et al. Gene deletions and amplifications in human hepatocellular carcinomas: correlation with hepatocyte growth regulation. The Americanjournal ofpathology 180, 1495-1508 (2012).
8. Chen, Z. H. et al. MAN2A1-FER Fusion Gene Is Expressed by Human Liver and Other Tumor Types and Has Oncogenic Activity in Mice. Gastroenterology 153, 1120-1132, doi:10.1053/j.gastro.2016.12.036 (2017).
9. He, D. M. et al. Oncogenic activity of amplified miniature chromosome maintenance 8 in human malignancies. Oncogene 36, 3629-3639, doi:10.1038/onc.2017.123 (2017).
10. Yu, Y. P. et al. Identification of recurrent fusion genes across multiple cancer types. Sci Rep 9, 1074, doi:10.1038/s41598-019-38550-6 (2019).
11. Yu, Y. P. et al. Detection of fusion transcripts in the serum samples of patients with hepatocellular carcinoma. Oncotarget 10, 3352-3360 (2019).
12. Liu, S. et al. Transcriptome and Exome Analyses of Hepatocellular Carcinoma Reveal Patterns to Predict Cancer Recurrence in Liver Transplant Patients. Hepatol Commun, doi:10.1002/hep4.1846 (2021).
13. Luo, J. H. et al. Pten-NOLC1 fusion promotes cancers involving MET and EGFR signalings. Oncogene 40, 1064-1076, doi:10.1038/s41388-020-01582-8 (2021).
14. Chen, Z. H. et al. Targeting genomic rearrangements in tumor cells through Cas9-mediated insertion of a suicide gene. Nature biotechnology 35, 543-550, doi:10.1038/nbt.3843 (2017).
15. Wong, C. M., Fan, S. T. & Ng, I. O. beta-Catenin mutation and overexpression in hepatocellular carcinoma: clinicopathologic and prognostic significance. Cancer 92, 136-145, doi:10.1002/1097-0142(20010701)92:1<136::aid-cncrl301>3.0.co;2-r (2001).
16. Nejak-Bowen, K. N. et al. Accelerated liver regeneration and hepatocarcinogenesis in mice overexpressing serine-45 mutant beta-catenin. Hepatology (Baltimore, Md 51, 1603-1613, doi:10.1002/hep.23538 (2010).
17. Ze-Hua Zuo et al. Oncogenic activity of SLC45A2-AMACR gene fusion is mediated by mitogen-activated protein kinase. Hepatology Communications in press (2021).18. Kim, W. et al. Targeting mutant KRAS with CRISPR-Cas9 controls tumor growth. Genome research
28, 374-382, doi:10.1101/gr.223891.117 (2018). 19. Wang, H. et al. p53-induced gene 3 mediates cell death induced by glutathione peroxidase 3. The Journal of biological chemistry 287, 16890-16902 (2012).
20. Zhu, Z. H. et al. Integrin alpha 7 interacts with high temperature requirement A2 (HtrA2) to induce prostate cancer cell death. The American journal ofpathology 177, 1176-1186 (2010).
21. Shi, Y. K., Yu, Y. P., Tseng, G. C. & Luo, J. H. Inhibition of prostate cancer growth and metastasis using small interference RNA specific for minichromosome complex maintenance component 7. Cancer gene therapy 17, 694-699 (2010).
22. Luo, K. L., Luo, J. H. & Yu, Y. P. (-)-Epigallocatechin-3-gallate induces Du145 prostate cancer cell death via downregulation of inhibitor of DNA binding 2, a dominant negative helix-loop-helix protein. Cancer science 101, 707-712 (2010).
23. Han, Y. C. et al. Interaction of integrin-linked kinase and miniature chromosome maintenance 7—mediating integrin {alpha}7 induced cell growth suppression. Cancer research 70, 4375-4384 (2010).
24. Zhu, Z. H., Yu, Y. P., Shi, Y. K., Nelson, J. B. & Luo, J. H. CSR1 induces cell death through inactivation of CPSF3. Oncogene 28, 41-51 (2009).
25. Yu, Y. P. et al. Glutathione peroxidase 3, deleted or methylated in prostate cancer, suppresses prostate cancer growth and metastasis. Cancer research 67, 8043-8050 (2007).
26. Ren, B. et al. Analysis of integrin alpha7 mutations in prostate cancer, liver cancer, glioblastoma multiforme, and leiomyosarcoma. Journal of the National Cancer Institute 99, 868-880 (2007).
27. Yu, G. et al. CSR1 suppresses tumor growth and metastasis of prostate cancer. American Journal of Pathology 168, 597-607 (2006).
28. Liu, F., Song, Y. & Liu, D. Hydrodynamics-based transfection in animals by systemic administration of plasmid DNA. Gene therapy 6, 1258-1266 (1999).
29. Tao, J. et al. Modeling a Human HCC Subset in Mice Through Co-Expression of Met and Point-Mutant beta-Catenin. Hepatology (Baltimore, Md (2016).
30. Han, Y. C. et al. Metallothionein 1 h tumour suppressor activity in prostate cancer is mediated by euchromatin methyltransferase 1. The Journal of pathology 230, 184-193 (2013).
31. Ren, B. et al. MCM7 amplification and overexpression are associated with prostate cancer progression. Oncogene 25, 1090-1098 (2006).
32. Jing, L. et al. Expression of myopodin induces suppression of tumor growth and metastasis. The American journal of pathology 164, 1799-1806 (2004).

Although the presently disclosed subject matter and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps.

Patents, patent applications, publications, product descriptions and protocols are cited throughout this application the disclosures of which are incorporated herein by reference in their entireties for all purposes.

What is claimed is:

1. A genome editing method comprising:
(i) introducing a first polynucleotide encoding a suicide gene and a second polynucleotide encoding a nuclease into a cell, wherein the nuclease targets a genomic mutation of a gene selected from CTNNB1, SLTM, SLC45A2, AMACR, or a combination thereof, and
(ii) contacting the cell with an agent capable of inducing killing of the cell.

2. The genome editing method of claim 1, wherein the suicide gene is selected from herpes simplex virus thymidine kinase (HSV-tk), Exotoxin A from Pseudomonas aeruginosa, Diphtheria toxin from Corynebacterium diphtheri, Ricin or abrin from Ricinus communi (castor oil plant), Cytosine deaminase, Carboxyl esterase, Varicella Zoster virus (VZV) thymidine kinase, or a combination thereof.

3. The genome editing method of claim 2, wherein the suicide gene comprises an amino acid sequence at least 80% identical to the amino acid sequence set forth in SEQ ID NO: 25.

4. The genome editing method of claim 1, wherein the first polynucleotide comprises a first homology recombination arm and a second homology recombination arm, wherein the first homology recombination arm and the second homology recombination arm flank the first polynucleotide.

5. The genome editing method of claim 1, wherein the nuclease is Cas9 or Cas9^{D10A}.

6. The genome editing method of claim 1, further comprising one or more guide RNAs (gRNAs), wherein the one or more gRNAs target the genomic mutation selected from:
(i) a S45P mutation of the CTNNB1 gene;
(ii) a V235G mutation of the SLTM gene;
(iii) a mutation of a breakpoint of a SLC45A2-AMACR fusion gene; or
(iv) a combination thereof.

7. The genome editing method of claim 1, wherein the first polynucleotide and the second polynucleotide are included in a single vector, or wherein the first polynucleotide is included in a first vector and the second polynucleotide is included in a second vector.

8. The genome editing method of claim 1, wherein the agent is selected from ganciclovir, valganciclovir, or a combination thereof.

9. A genome editing system comprising:
(i) a first polynucleotide encoding a suicide gene;
(ii) a second polynucleotide encoding a nuclease targeting a genomic mutation of a gene selected from CTNNB1, SLTM, SLC45A2, AMACR, or a combination thereof; and
(iii) a therapeutically effective amount of an agent to induce the killing of a cell expressing the suicide gene.

10. The genome editing system of claim 9, wherein the suicide gene is selected from herpes simplex virus thymidine kinase (HSV-tk), inducible Caspase 9 suicide gene (iCasp-9), truncated human epidermal growth factor receptor (EGFRt), or a combination thereof.

11. The genome editing system of claim 10, wherein the suicide gene comprises an amino acid sequence at least 80% identical to the amino acid sequence set forth in SEQ ID NO: 25.

12. The genome editing system of claim 9, wherein the first polynucleotide comprises a first homology recombination arm and a second homology recombination arm, wherein the first homology recombination arm and the second homology recombination arm flank the first polynucleotide.

13. The genome editing system of claim 9, wherein the nuclease is Cas9 or Cas9^{D10A}.

14. The genome editing system of claim 9, further comprising one or more guide RNAs (gRNAs), wherein the one or more gRNAs target the genomic mutation selected from:
(i) a S45P mutation of the CTNNB1 gene;
(ii) a V235G mutation of the SLTM gene;
(iii) a mutation of a breakpoint of a SLC45A2-AMACR fusion gene; or
(iv) a combination thereof.

15. The genome editing system of claim 9, wherein the first polynucleotide and the second polynucleotide are included in a single vector, or wherein the first polynucleotide is included in a first vector and the second polynucleotide is included in a second vector.

16. The genome editing system of claim 9, wherein the agent is selected from ganciclovir, valganciclovir, or a combination thereof.

17. A method of treating a subject having a pre-malignant or neoplastic condition, the method comprising:
(i) detecting, in a sample obtained from the subject, a genomic mutation of a gene selected from CTNNB1, SLTM, SLC45A2, AMACR, or a combination thereof,
(ii) administering an effective amount of a first polynucleotide encoding a suicide gene and a second polynucleotide encoding a nuclease targeting the genomic mutation; and
(iii) administering to the subject an effective amount of an agent to induce the killing of a cell expressing the suicide gene.

18. The method of claim 17, wherein the pre-malignant or neoplastic condition is a condition of the liver.

19. The method of claim 17, wherein the suicide gene is selected from herpes simplex virus thymidine kinase (HSV-tk), Exotoxin A from Pseudomonas aeruginosa, Diphtheria toxin from Corynebacterium diphtheri, Ricin or abrin from Ricinus communi (castor oil plant), Cytosine deaminase, Carboxyl esterase, Varicella Zoster virus (VZV) thymidine kinase, or a combination thereof, and wherein the nuclease is Cas9 or Cas9^{D10A}.

20. The method of claim 17, further comprising one or more guide RNAs (gRNAs), wherein the one or more gRNAs target the genomic mutation selected from:
(i) a S45P mutation of the CTNNB1 gene;
(ii) a V235G mutation of the SLTM gene;
(iii) a mutation of a breakpoint of a SLC45A2-AMACR fusion gene; or
(iv) a combination thereof.


SEQUENCE LISTING (27 sequences)

SEQ ID NO: 1 - DNA, 43 bp, synthetic construct
  misc_feature 1..43: note = Description of sequence: Primer
  source 1..43: mol_type = other DNA
gtcgaccaccatggagcaaaagctcatttctgaagaggacttg

SEQ ID NO: 2 - DNA, 27 bp, synthetic construct
  misc_feature 1..27: note = Description of sequence: Primer
  source 1..27: mol_type = other DNA
gcggccgcttacaggtcagtatcaaac

SEQ ID NO: 3 - DNA, 28 bp, synthetic construct
  misc_feature 1..28: note = Description of sequence: Primer
  source 1..28: mol_type = other DNA
tttaaaaggatacagcggcttctgcgcg

SEQ ID NO: 4 - DNA, 27 bp, synthetic construct
  misc_feature 1..27: note = Description of sequence: Primer
  source 1..27: mol_type = other DNA
gtcgaccacgctggattttcaaaacag

SEQ ID NO: 5 - DNA, 33 bp, synthetic construct
  misc_feature 1..33: note = Description of sequence: Primer
  source 1..33: mol_type = other DNA
gcctttaccactcagaggaggagctgtggtagt

SEQ ID NO: 6 - DNA, 38 bp, synthetic construct
  misc_feature 1..38: note = Description of sequence: Primer
  source 1..38: mol_type = other DNA
cactaccacagctcctcctctgagtggtaaaggcaatc

SEQ ID NO: 7 - DNA, 58 bp, synthetic construct
  misc_feature 1..58: note = Description of sequence: Primer
  source 1..58: mol_type = other DNA
ggccagtgaattgtaatacgactcactatagggaggcggaaaggcaatcctgaggaag

SEQ ID NO: 8 - DNA, 31 bp, synthetic construct
  misc_feature 1..31: note = Description of sequence: Primer
  source 1..31: mol_type = other DNA
aaaaaaagcaccgactcggtgccactttttc

SEQ ID NO: 9 - DNA, 60 bp, synthetic construct
  misc_feature 1..60: note = Description of sequence: Primer
  source 1..60: mol_type = other DNA
ggccagtgaattgtaatacgactcactatagggaggcggtctaccacagctcctcctctg

SEQ ID NO: 10 - DNA, 59 bp, synthetic construct
  misc_feature 1..59: note = Description of sequence: Primer
  source 1..59: mol_type = other DNA
ggccagtgaattgtaatacgactcactatagggaggcggggtgctaaactttttcgtga

SEQ ID NO: 11 - DNA, 59 bp, synthetic construct
  misc_feature 1..59: note = Description of sequence: Primer
  source 1..59: mol_type = other DNA
ggccagtgaattgtaatacgactcactatagggaggcgggagagctcccattttcctcc

SEQ ID NO: 12 - DNA, 59 bp, synthetic construct
  misc_feature 1..59: note = Description of sequence: Primer
  source 1..59: mol_type = other DNA
ggccagtgaattgtaatacgactcactatagggaggcggctgtgtgatcagcctcagct

SEQ ID NO: 13 - DNA, 59 bp, synthetic construct
  misc_feature 1..59: note = Description of sequence: Primer
  source 1..59: mol_type = other DNA
ggccagtgaattgtaatacgactcactatagggaggcggagatggaagctaatgcgact

SEQ ID NO: 14 - DNA, 21 bp, synthetic construct
  misc_feature 1..21: note = Description of sequence: Primer
  source 1..21: mol_type = other DNA
ttgatgtctgctcccatcagg

SEQ ID NO: 15 - DNA, 21 bp, synthetic construct
  misc_feature 1..21: note = Description of sequence: Primer
  source 1..21: mol_type = other DNA
cagctggagtttctccatgac

SEQ ID NO: 16 - DNA, 21 bp, synthetic construct
  misc_feature 1..21: note = Description of sequence: Primer
  source 1..21: mol_type = other DNA
gccaggaaaatgggagctctc

SEQ ID NO: 17 - DNA, 21 bp, synthetic construct
  misc_feature 1..21: note = Description of sequence: Primer
  source 1..21: mol_type = other DNA
tccctatcagctcatgagctc

SEQ ID NO: 18 - protein, 376 aa, synthetic construct
  REGION 1..376: note = Description of sequence: Peptide: HSV-tk
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 19 - protein, 376 aa, synthetic construct
  REGION 1..376: note = Description of sequence: Peptide: HSV-tk
MASHAGQQHAPAFGQAARASGPTDGRAASRPSHRQGASEARGDPELPTLLRVYIDGPHGVGKTTTSAQLMEALGPRDNIVYVPEPMTYWQVLGASETLTNIYNTQHRLDRGEISAGEAAVVMTSAQITMSTPYAATDAVLAPHIGGEAVGPQAPPPALTLVFDRHPIASLLCYPAARYLMGSMTPQAVLAFVALMPPTAPGTNLVLGVLPEAEHADRLARRQRPGERLDLAMLSAIRRVYDLLANTVRYLQRGGRWREDWGRLTGVAAATPRPDPEDGAGSLPRIEDTLFALFRVPELLAPNGDLYHIFAWVLDVLADRLLPMHLFVLDYDQSPVGCRDALLRLTAGMIPTRVTTAGSIAEIRDLARTFAREVGGV

SEQ ID NO: 20 - protein, 376 aa, synthetic construct
  REGION 1..376: note = Description of sequence: Peptide: HSV-tk
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 21 - protein, 376 aa, synthetic construct
  REGION 1..376: note = Description of sequence: Peptide: HSV-tk
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARMFAREMGEAN

SEQ ID NO: 22 - protein, 376 aa, synthetic construct
  REGION 1..376: note = Description of sequence: Peptide: HSV-tk
MASYPGHQHASAFDQAARSRGHSNRRTALRPRRQQEATEVRPEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMIQTHVTTPGSIPTICDLARTFAREMGEAN

SEQ ID NO: 23 - protein, 376 aa, synthetic construct; identical to SEQ ID NO: 21
  REGION 1..376: note = Description of sequence: Peptide: HSV-tk

SEQ ID NO: 24 - protein, 376 aa, synthetic construct
  REGION 1..376: note = Description of sequence: Peptide: HSV-tk
MASHAGQQHAPAFGQAARASGPTDGRAASRPSHRQGASGARGDPELPTLLRVYIDGPHGVGKTTTSAQLMEALGPRDNIVYVPEPMTYWQVLGASETLTNIYNTQHRLDRGEISAGEAAVVMTSAQITMSTPYAATDAVLAPHIGGEAVGPQAPPPALTLVFDRHPIASLLCYPAARYLMGSMTPQAVLAFVALMPPTAPGTNLVLGVLPEAEHADRLARRQRPGERLDLAMLSAIRRVYDLLANTVRYLQRGGRWREDWGRLTGVAAATPRPDPEDGAGSLPRIEDTLFALFRVPELLAPNGDLYHIFAWVLDVLADRLLPMHLFVLDYDQSPVGCRDALLRLTAGMIPTRVTTAGSIAEIRDLARTFAREVGGV

SEQ ID NO: 25 - protein, 376 aa, synthetic construct
  REGION 1..376: note = Description of sequence: Peptide: HSV-tk
MASYPGHQHASAFDQAARSRGHSNRRTALRPRRQQEATEVRPEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQCGGSWREDWGQLSGTAVPPQGAEPQSNAGPRPHIGDTLFTLFRAPELLAPNGDLYNVFAWALDVLAKRLRSMHVFILDYDQSPAGCRDALLQLTSGMVQTHVTTPGSIPTICDLARTFAREMGEAN

SEQ ID NO: 26 - DNA, 51 bp, synthetic construct
  misc_feature 1..51: note = Description of sequence: wild type exon 2 of CTNNB1
  source 1..51: mol_type = other DNA
ccactaccacagctcctcctctgagtggtaaaggcaatcctgaggaagagg

SEQ ID NO: 27 - DNA, 51 bp, synthetic construct
  misc_feature 1..51: note = Description of sequence: mutation from exon 2 of CTNNB1
  source 1..51: mol_type = other DNA
ccactaccacagctccttctctgagtggtaaaggcaatcctgaggaagagg